SEQUENCE LISTING (102 sequences)

SEQ ID NO: 1 - DNA, 12 bp, Artificial Sequence
  Chemically synthesized oligonucleotide
gtggcgattatc

SEQ ID NO: 2 - DNA, 12 bp, Artificial Sequence
  Chemically synthesized oligonucleotide
ttagtatccgtc

SEQ ID NO: 3 - DNA, 12 bp, Artificial Sequence
  Chemically synthesized oligonucleotide
tcagtaactgcg

SEQ ID NO: 4 - DNA, 12 bp, Artificial Sequence
  Chemically synthesized oligonucleotide
gtggcgattatc

SEQ ID NO: 5 - DNA, 12 bp, Artificial Sequence
  Chemically synthesized oligonucleotide
ttagtatccgtc

SEQ ID NO: 6 - DNA, 12 bp, Artificial Sequence
  Chemically synthesized oligonucleotide
tcagtaactgcg

SEQ ID NO: 7 - DNA, 13 bp, Artificial Sequence
  Chemically synthesized oligonucleotide
ctgggcattactg

SEQ ID NO: 8 - DNA, 13 bp, Artificial Sequence
  Chemically synthesized oligonucleotide
atagattccgctg

SEQ ID NO: 9 - DNA, 13 bp, Artificial Sequence
  Chemically synthesized oligonucleotide
acagatactgtcc

SEQ ID NO: 10 - DNA, 10 bp, Artificial Sequence
  Chemically synthesized oligonucleotide
ctcattactg

SEQ ID NO: 11 - DNA, 10 bp, Artificial Sequence
  Chemically synthesized oligonucleotide
atttccgctg

SEQ ID NO: 12 - DNA, 10 bp, Artificial Sequence
  Chemically synthesized oligonucleotide
actactgtcc

SEQ ID NO: 13 - DNA, 5 bp, Artificial Sequence
  Chemically synthesized oligonucleotide
cagat

SEQ ID NO: 14 - DNA, 5 bp, Artificial Sequence
  Chemically synthesized oligonucleotide
tagat

SEQ ID NO: 15 - DNA, 4 bp, Artificial Sequence
  Chemically synthesized oligonucleotide
ctgc

SEQ ID NO: 16 - DNA, 4 bp, Artificial Sequence
  Chemically synthesized oligonucleotide
ccgt

SEQ ID NO: 17 - DNA, 4 bp, Artificial Sequence
  Chemically synthesized oligonucleotide
ttcg

SEQ ID NO: 18 - DNA, 4 bp, Artificial Sequence
  Chemically synthesized oligonucleotide
cttg

SEQ ID NO: 19 - DNA, 12 bp, Artificial Sequence
  Chemically synthesized oligonucleotide
atagattccgtg

SEQ ID NO: 20 - DNA, 5 bp, Artificial Sequence
  Chemically synthesized oligonucleotide
tagat

SEQ ID NO: 21 - DNA, 12 bp, Artificial Sequence
  Chemically synthesized oligonucleotide
gtggcgattatc

SEQ ID NO: 22 - DNA, 12 bp, Artificial Sequence
  Chemically synthesized oligonucleotide
tcagtaactgcg

SEQ ID NO: 23 - DNA, 12 bp, Artificial Sequence
  Chemically synthesized oligonucleotide
ttagtaactgcg

SEQ ID NO: 24 - DNA, 12 bp, Artificial Sequence
  Chemically synthesized oligonucleotide
ttagtatccgtc

SEQ ID NO: 25 - DNA, 12 bp, Artificial Sequence
  Chemically synthesized oligonucleotide
ttgacatccgtc

SEQ ID NO: 26 - DNA, 12 bp, Artificial Sequence
  Chemically synthesized oligonucleotide
gtgacatccgtc

SEQ ID NO: 27 - DNA, 12 bp, Artificial Sequence
  Chemically synthesized oligonucleotide
gtggcgattatg

SEQ ID NO: 28 - DNA, 12 bp, Artificial Sequence
  Chemically synthesized oligonucleotide
tcagtaattatc

SEQ ID NO: 29 - DNA, 12 bp, Artificial Sequence
  Chemically synthesized oligonucleotide
ttagtatctgcg

SEQ ID NO: 30 - DNA, 12 bp, Artificial Sequence
  Chemically synthesized oligonucleotide
tcagtaactgcc

SEQ ID NO: 31 - DNA, 12 bp, Artificial Sequence
  Chemically synthesized oligonucleotide
ttagcgattatc

SEQ ID NO: 32 - DNA, 11 bp, Artificial Sequence
  Chemically synthesized oligonucleotide
gtggcgatatc

SEQ ID NO: 33 - DNA, 11 bp, Artificial Sequence
  Chemically synthesized oligonucleotide
tcagtaatgcg

SEQ ID NO: 34 - DNA, 11 bp, Artificial Sequence
  Chemically synthesized oligonucleotide
ttagtaatgcg

SEQ ID NO: 35 - DNA, 11 bp, Artificial Sequence
  Chemically synthesized oligonucleotide
ttagtatcgtc

SEQ ID NO: 36 - DNA, 11 bp, Artificial Sequence
  Chemically synthesized oligonucleotide
ttgacatcgtc

SEQ ID NO: 37 - DNA, 11 bp, Artificial Sequence
  Chemically synthesized oligonucleotide
gtgacatcgtc

SEQ ID NO: 38 - DNA, 11 bp, Artificial Sequence
  Chemically synthesized oligonucleotide
tcagtaatatc

SEQ ID NO: 39 - DNA, 11 bp, Artificial Sequence
  Chemically synthesized oligonucleotide
gtggcgatatg

SEQ ID NO: 40 - DNA, 11 bp, Artificial Sequence
  Chemically synthesized oligonucleotide
ttagtattatc

SEQ ID NO: 41 - DNA, 11 bp, Artificial Sequence
  Chemically synthesized oligonucleotide
ttagtattgcg

SEQ ID NO: 42 - DNA, 11 bp, Artificial Sequence
  Chemically synthesized oligonucleotide
tcagtaatgcc

SEQ ID NO: 43 - DNA, 11 bp, Artificial Sequence
  Chemically synthesized oligonucleotide
ttagcgatatc

SEQ ID NO: 44 - DNA, 10 bp, Artificial Sequence
  Chemically synthesized oligonucleotide
gtggcgtatc

SEQ ID NO: 45 - DNA, 10 bp, Artificial Sequence
  Chemically synthesized oligonucleotide
tcagtatgcg

SEQ ID NO: 46 - DNA, 10 bp, Artificial Sequence
  Chemically synthesized oligonucleotide
ttagtatgcg

SEQ ID NO: 47 - DNA, 10 bp, Artificial Sequence
  Chemically synthesized oligonucleotide
ttagtacgtc

SEQ ID NO: 48 - DNA, 10 bp, Artificial Sequence
  Chemically synthesized oligonucleotide
ttgacacgtc

SEQ ID NO: 49 - DNA, 10 bp, Artificial Sequence
  Chemically synthesized oligonucleotide
gtgacacgtc

SEQ ID NO: 50 - DNA, 10 bp, Artificial Sequence
  Chemically synthesized oligonucleotide
tcagtatatc

SEQ ID NO: 51 - DNA, 10 bp, Artificial Sequence
  Chemically synthesized oligonucleotide
gtggcgtatg

SEQ ID NO: 52 - DNA, 10 bp, Artificial Sequence
  Chemically synthesized oligonucleotide
ttagtatatc

SEQ ID NO: 53 - DNA, 10 bp, Artificial Sequence
  Chemically synthesized oligonucleotide
tcagtatgcc

SEQ ID NO: 54 - DNA, 10 bp, Artificial Sequence
  Chemically synthesized oligonucleotide
ttagcgtatc

SEQ ID NO: 55 - DNA, 9 bp, Artificial Sequence
  Chemically synthesized oligonucleotide
gggcgtatc

SEQ ID NO: 56 - DNA, 9 bp, Artificial Sequence
  Chemically synthesized oligonucleotide
tagtatgcg

SEQ ID NO: 57 - DNA, 9 bp, Artificial Sequence
  Chemically synthesized oligonucleotide
tagtacgtc

SEQ ID NO: 58 - DNA, 9 bp, Artificial Sequence
  Chemically synthesized oligonucleotide
ggacacgtc

SEQ ID NO: 59 - DNA, 9 bp, Artificial Sequence
  Chemically synthesized oligonucleotide
tgacacgtc

SEQ ID NO: 60 - DNA, 9 bp, Artificial Sequence
  Chemically synthesized oligonucleotide
gagtatatc

SEQ ID NO: 61 - DNA, 9 bp, Artificial Sequence
  Chemically synthesized oligonucleotide
tagtatatc

SEQ ID NO: 62 - DNA, 9 bp, Artificial Sequence
  Chemically synthesized oligonucleotide
gggcgtatg

SEQ ID NO: 63 - DNA, 9 bp, Artificial Sequence
  Chemically synthesized oligonucleotide
tagtatgcc

SEQ ID NO: 64 - DNA, 9 bp, Artificial Sequence
  Chemically synthesized oligonucleotide
tagcgtatc

SEQ ID NO: 65 - DNA, 8 bp, Artificial Sequence
  Chemically synthesized oligonucleotide
gggcgatc

SEQ ID NO: 66 - DNA, 8 bp, Artificial Sequence
  Chemically synthesized oligonucleotide
tagtagcg

SEQ ID NO: 67 - DNA, 8 bp, Artificial Sequence
  Chemically synthesized oligonucleotide
tagtagtc

SEQ ID NO: 68 - DNA, 8 bp, Artificial Sequence
  Chemically synthesized oligonucleotide
ggacagtc

SEQ ID NO: 69 - DNA, 8 bp, Artificial Sequence
  Chemically synthesized oligonucleotide
tgacagtc

SEQ ID NO: 70 - DNA, 8 bp, Artificial Sequence
  Chemically synthesized oligonucleotide
gagtaatc

SEQ ID NO: 71 - DNA, 8 bp, Artificial Sequence
  Chemically synthesized oligonucleotide
tagtaacg

SEQ ID NO: 72 - DNA, 8 bp, Artificial Sequence
  Chemically synthesized oligonucleotide
tagtaatc

SEQ ID NO: 73 - DNA, 8 bp, Artificial Sequence
  Chemically synthesized oligonucleotide
gggcgatg

SEQ ID NO: 74 - DNA, 8 bp, Artificial Sequence
  Chemically synthesized oligonucleotide
tagtagcc

SEQ ID NO: 75 - DNA, 8 bp, Artificial Sequence
  Chemically synthesized oligonucleotide
tagcgatc

SEQ ID NO: 76 - DNA, 7 bp, Artificial Sequence
  Chemically synthesized oligonucleotide
gggcgat

SEQ ID NO: 77 - DNA, 7 bp, Artificial Sequence
  Chemically synthesized oligonucleotide
tagtagc

SEQ ID NO: 78 - DNA, 7 bp, Artificial Sequence
  Chemically synthesized oligonucleotide
tagtagt

SEQ ID NO: 79 - DNA, 7 bp, Artificial Sequence
  Chemically synthesized oligonucleotide
tgacagt

SEQ ID NO: 80 - DNA, 7 bp, Artificial Sequence
  Chemically synthesized oligonucleotide
ggacagt

SEQ ID NO: 81 - DNA, 7 bp, Artificial Sequence
  Chemically synthesized oligonucleotide
gagtaat

SEQ ID NO: 82 - DNA, 7 bp, Artificial Sequence
  Chemically synthesized oligonucleotide
tagtaac

SEQ ID NO: 83 - DNA, 7 bp, Artificial Sequence
  Chemically synthesized oligonucleotide
tagtaat

SEQ ID NO: 84 - DNA, 7 bp, Artificial Sequence
  Chemically synthesized oligonucleotide
tagcgat

SEQ ID NO: 85 - DNA, 6 bp, Artificial Sequence
  Chemically synthesized oligonucleotide
tgtagc

SEQ ID NO: 86 - DNA, 6 bp, Artificial Sequence
  Chemically synthesized oligonucleotide
ggcgat

SEQ ID NO: 87 - DNA, 6 bp, Artificial Sequence
  Chemically synthesized oligonucleotide
tgtagt

SEQ ID NO: 88 - DNA, 6 bp, Artificial Sequence
  Chemically synthesized oligonucleotide
tacagt

SEQ ID NO: 89 - DNA, 6 bp, Artificial Sequence
  Chemically synthesized oligonucleotide
tgcgat

SEQ ID NO: 90 - DNA, 6 bp, Artificial Sequence
  Chemically synthesized oligonucleotide
ggtaat

SEQ ID NO: 91 - DNA, 6 bp, Artificial Sequence
  Chemically synthesized oligonucleotide
tgtaat

SEQ ID NO: 92 - DNA, 6 bp, Artificial Sequence
  Chemically synthesized oligonucleotide
gacagt

SEQ ID NO: 93 - DNA, 5 bp, Artificial Sequence
  Chemically synthesized oligonucleotide
gtagc

SEQ ID NO: 94 - DNA, 5 bp, Artificial Sequence
  Chemically synthesized oligonucleotide
gcgat

SEQ ID NO: 95 - DNA, 5 bp, Artificial Sequence
  Chemically synthesized oligonucleotide
gtagt

SEQ ID NO: 96 - DNA, 5 bp, Artificial Sequence
  Chemically synthesized oligonucleotide
acagt

SEQ ID NO: 97 - DNA, 5 bp, Artificial Sequence
  Chemically synthesized oligonucleotide
gtaat

SEQ ID NO: 98 - DNA, 4 bp, Artificial Sequence
  Chemically synthesized oligonucleotide
gagc

SEQ ID NO: 99 - DNA, 4 bp, Artificial Sequence
  Chemically synthesized oligonucleotide
ggat

SEQ ID NO: 100 - DNA, 4 bp, Artificial Sequence
  Chemically synthesized oligonucleotide
aagt

SEQ ID NO: 101 - DNA, 4 bp, Artificial Sequence
  Chemically synthesized oligonucleotide
gagt

SEQ ID NO: 102 - DNA, 4 bp, Artificial Sequence
  Chemically synthesized oligonucleotide
gaat

(19) United States
(12) Patent Application Publication
(10) Pub. No.: US 2012/0115137 A1
(43) Pub. Date: May 10, 2012

(54) Methods of Predicting Osteoarthritis

(76) Inventors: Kenneth S. Kornman, Newton, MA (US); Xiaodong Wu, (US); Venkateswarlu Kondragunta, Fishers, IN (US); Joanne Jordan, (US); Gordon W. Duff, Sheffield (GB)

(21) Appl. No.: 13/222,486

(22) Filed: Aug. 31, 2011

Related U.S. Application Data
(60) Provisional application No. 61/378,908, filed on Aug. 31, 2010.

(51) Int. Cl. C12Q 1/68

(52) U.S. Cl. 435/6.11

(57) ABSTRACT

The present invention relates generally to methods for predicting progression, initiation and susceptibility of osteoarthritis in human subjects using their genotype test results.

### RELATED APPLICATIONS

This application claims the priority to the U.S. Provisional Application No. 61/378,908, filed Aug. 31, 2010, which is incorporated herein by reference in its entirety.

### FIELD OF THE INVENTION

This invention relates to methods and kits for detecting a predisposition to, determining risk of, and guiding therapy for osteoarthritis progression, osteoarthritis initiation, and susceptibility to osteoarthritis.

### BACKGROUND

Osteoarthritis (OA) is a chronic joint disorder and is generally considered a degenerative disease of aging, and the incidence rises with age. The etiology of osteoarthritis is multifactorial involving both mechanical and biochemical factors. Primary osteoarthritis generally refers to osteoarthritis of no known cause. Secondary osteoarthritis generally refers to osteoarthritis resulting from some external or internal injury or disease (obesity, repeated trauma or surgery to the joint structures, abnormal joints at birth (congenital abnormalities), gout, diabetes and other hormone disorders). Generalized osteoarthritis affects many joints. Localized osteoarthritis typically affects a single joint, though in some cases, such as with finger arthritis, several joints may be affected. Osteoarthritis affects 5-20% of world's population and increasing in frequency and severity in all aging populations. The estimated U.S. prevalence is 15-60 million patients; 300-1200 million worldwide. These numbers are expected to increase 525% by 2030. Currently there is no FDA-approved therapy that arrests or reverses the joint deterioration.

Given the anticipated increase in osteoarthritis prevalence, there is a need to optimize the management of osteoarthritis and to increase our knowledge regarding the predictors of osteoarthritis progression, initiation and susceptibility. Such prognostic factors may be used to identify high-risk groups for the development (or onset) of OA and/or high-risk groups for the severe disease progression of OA. These prognostic factors may also help to develop new drugs which prevent or treat osteoarthritis in high risk groups.

### BRIEF SUMMARY OF THE INVENTION

One aspect of the invention is directed to a method for predicting progression of osteoarthritis in a patient, comprising the steps of: (a) taking a biological sample from said patient; (b) genotyping said biological sample for (i) at least one of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1(rs2287047), IL1R1 (rs315949), VDR (rs10735810), SLC17A3 (rs1165205), OPG (rs2073618), Cilp (rs2073711) and IL1RN (rs4251961) and (ii) optionally one or more genetic markers selected from the group consisting of IL1RN (rs419598), IL1RN (rs315931), IL1RN (rs3181052), IL1RN (rs579543) and IL1RN (rs9005); (c) comparing the genotyping results of step b with a reference; and (d) predicting progress of osteoarthritis of said patient based on the patient's genotype. Preferably the biological sample is genotyped for at least two, three, four, five, six, seven or eight markers and even more preferably at least nine, ten, eleven, twelve, thirteen, fourteen, fifteen, sixteen, seventeen, eighteen, nineteen, twenty or twenty-one markers.

Another aspect of the invention is directed to a method for predicting initiation of osteoarthritis in a patient, comprising the steps of (a) taking a biological sample from said patient; (b) genotyping said biological sample for at least one of the genetic markers selected from the group consisting of ADAM12 (rs3740199), BMP2 (rs1049007), CLEC3B (rs13963), HFE (rs1799945), IL1RN (rs315931), IL1RN (rs419598), IL1RN (rs579543), IL1RN (rs9005), IL1B(rs1143623), ADAM12(rs1871054), OPG(rs2073618), IL1RN(rs315943), IL1RN(rs315949), IL1RN(rs4251961), CDC42BPB(rs751837) and IL1RN(rs315952); (c) comparing the genotyping results of step b with a reference; and (d) predicting said patient's risk of osteoarthritis initiation based on said patient's genotype. Preferably the biological sample is genotyped for at least two, three, four, five, six, seven or eight markers and even more preferably at least nine, ten, eleven, twelve, thirteen, fourteen, fifteen or sixteen markers.

Another aspect of the invention is directed to a method for predicting a patient's susceptibility to osteoarthritis, comprising the steps of (a) taking a biological sample from said patient; (b) genotyping said biological sample for (i) at least one of the genetic markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236) and (ii) optionally IL1RN(rs315931), IL1RN (rs4251961), IL1RN (rs2637988), IL1RN (rs3181052), IL (rs1794066), IL (rs419598), IL (rs380092), IL (rs579543), IL1RN(rs9005), IL1RN(rs315943) and IL1RN(rs1374281); (c) comparing the genotyping results of step b with a reference; (d) predicting initiation of osteoarthritis of said patient based on the patient's genotype. Preferably the biological sample is genotyped for at least two, three, four, five, six, seven or eight markers and even more preferably at least nine, ten, eleven, twelve, thirteen, fourteen, fifteen, sixteen, seventeen, eighteen, nineteen, twenty or twenty-one, twenty-two or twenty-three markers.

Another aspect of the invention is directed to a method of distinguishing human subjects having joint measurements of grade 0 on KL scale with those having joint measurements of grade 1, comprising the steps of (a) taking a biological sample from said patient; (b) genotyping said biological sample for at least one of the genetic markers selected from the group consisting of ABCG2 (rs2231142), ADAM12 (rs3740199), DVWA (rs11718863), IL1 RN (rs419598), IL1 RN (rs579543), IL1 RN (rs9005), IL6 (rs1800797), and PHACTR2 (rs7757372); (c) comparing the genotyping results of step b with a reference; and (d) separating said human subjects into groups of grade 0 on LK scale and grade 1 on KL scale based on the patients' genotypes.

The contents of the patents and publications cited herein and the contents of documents cited in these patents and publications are hereby incorporated herein by reference to the extent permitted.

### BRIEF DESCRIPTION OF THE DRAWINGS

FIG. 1. Linkage disequilibrium (LD) map. The LD map was generated in Haploview software (D′ shown) for 13 IL1RN SNPs analyzed in the JoCo study.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

As used herein, the term “BMP2 (rs1049007)” means a single nucleotide polymorphism in the bone morphogenetic protein 2 (BMP2) gene. This is an A/G nucleotide substitution. The sequence surrounding this SNP is available from the dbSNP database of the National Center for Biotechnology Information www.ncbi.nlm.nih.gov/projects/SNP/snp_ref.cgi?rs=1049007);

The term “CLEC3B (rs13963)” means a single nucleotide polymorphism in the C-type lectin domain family 3, member B (CLEC3B) gene. This is an A/G nucleotide substitution. The sequence surrounding this SNP is available from the dbSNP database of the National Center for Biotechnology Information (www.ncbi.nlm.nih.gov/projects/SNP/snp_ref.cgi?rs=13963).

“IL1RN (rs1374281)” means a single nucleotide polymorphism in the interleukin 1 receptor antagonist (IL1RN) gene. This is a C/G nucleotide substitution. The sequence surrounding this SNP is available from the dbSNP database of the National Center for Biotechnology Information (www.ncbi.nlm.nih.gov/projects/SNP/snp_ref.cgi?rs=1374281);

“IL1RN (rs1794066)” means a single nucleotide polymorphism in the interleukin 1 receptor antagonist (IL1RN) gene. This is an AJG nucleotide substitution. The sequence surrounding this SNP is available from the dbSNP database of the National Center for Biotechnology (www.ncbi.nlm.nih.gov/projects/SNP/snp_ref.cgi?rs=1794066)

“IL1RN (rs2637988)” means a single nucleotide polymorphism in the interleukin 1 receptor antagonist (IL1RN) gene. This is an A/G nucleotide substitution. The sequence surrounding this SNP is available from the dbSNP database of the National Center for Biotechnology www.ncbi.nlm.nih.gov/projects/SNP/snp_ref.cgi?rs=2637988)

“IL1RN (rs315943)” means a single nucleotide polymorphism in the interleukin 1 receptor antagonist (IL1RN) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=315943);

“IL1RN (rs315952)” means a single nucleotide polymorphism in the interleukin 1 receptor antagonist (IL1RN) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=315952);

“IL1RN (rs380092)” means a single nucleotide polymorphism in the interleukin 1 receptor antagonist (IL1RN) gene. This is an A/T nucleotide substitution. The sequence surrounding this SNP is available from the dbSNP database of the National Center for Biotechnology (www.ncbi.nlm.nih.gov/projects/SNP/snp_ref.cgi?rs=380092).

“IL1RN (rs4251961)” means a single nucleotide polymorphism in the interleukin 1 receptor antagonist (IL1RN) gene. This is a C/Tnucleotide substitution. The sequence surrounding this SNP is available from the dbSNP database of the National Center for Biotechnology (www.ncbi.nlm.nih.gov/projects/SNP/snp_ref.cgi?rs=4251961).

“IL1R1 (rs2287047)” means a single nucleotide polymorphism in the interleukin 1 receptor, type I (IL1R1) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=2287047).

“rs315949” means a single nucleotide polymorphism with a C/T nucleotide substitution. The sequence surrounding this SNP is available from the dbSNP database of the National Center for Biotechnology (www.ncbi.nlm.nih.gov/projects/SNP/snp_ref.cgi?rs=315949).

As used herein, “ADAM12 (rs3740199)” means a single nucleotide polymorphism in the ADAM metallopeptidase domain 12 (ADAM12) gene. This is a C/G nucleotide substitution. The sequence surrounding this SNP is available from the dbSNP database of the National Center for Biotechnology (www.ncbi.nlm.nih.gov/projects/SNP/snp_ref.cgi?rs=3740199),

“HFE (rs1799945)” means a single nucleotide polymorphism in the hemochromatosis (HFE) gene. This is a C/G nucleotide substitution. The sequence surrounding this SNP is available from the dbSNP database of the National Center for Biotechnology (www.ncbi.nlm.nih.gov/projects/SNP/snp_ref.cgi?rs=1799945).

IL1RN (rs315931) means a single nucleotide polymorphism in the interleukin 1 receptor antagonist (IL1RN) gene. This is an A/C nucleotide substitution. The sequence surrounding this SNP is available from the dbSNP database of the National Center for Biotechnology (www.ncbi.nlm.nih.gov/projects/SNP/snp_ref.cgi?rs=315931),

“IL1RN (rs419598)” means a single nucleotide polymorphism in the interleukin 1 receptor antagonist (IL1RN) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=419598).

“IL1RN (rs579543)” means a single nucleotide polymorphism in the interleukin 1 receptor antagonist (IL1RN) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=579543).

IL1RN (rs9005) means a single nucleotide polymorphism in the interleukin 1 receptor antagonist (IL1RN) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=9005).

As used herein, “ABCG2(rs2231142)” means a single nucleotide polymorphism in the ATP-binding cassette, sub-family G (WHITE), member 2 (ABCG2) gene. This is an AJC nucleotide substitution. The sequence surrounding this SNP is available from the dbSNP database of the National Center for Biotechnology (www.ncbi.nlm.nih.gov/projects/SNP/snp_ref.cgi?rs=2231142).

ADAM12(rs3740199), DVWA(rs11718863) means a single nucleotide polymorphism with an A/T nucleotide substitution. The sequence surrounding this SNP is available from the dbSNP database of the National Center for Biotechnology (www.ncbi.nlm.nih.gov/projects/SNP/snp_ref.cgi?rs=11718863),

“ESR1(rs2234693)” means a single nucleotide polymorphism in the estrogen receptor I (ESR1) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=2234693).

“GDF5(rs143383)” means a single nucleotide polymorphism in the growth differentiation factor 5 (GDF5) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=143383).

“IL1A(rs10496444)” means a single nucleotide polymorphism with a C/T nucleotide substitution. The sequence surrounding this SNP is available from the dbSNP database of the National Center for Biotechnology (www.ncbi.nlm.nih.gov/projects/SNP/snp_ref.cgi?rs=10496444), IL1R1(rs2287047) means a single nucleotide polymorphism in the interleukin 1 receptor, type I (IL1R1) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=2287047).

“IL6(rs1800795)” means a single nucleotide polymorphism in the interleukin 6 (interferon, beta 2) gene. This is a C/G nucleotide substitution. The sequence surrounding this SNP is available from the dbSNP database of the National Center for Biotechnology (http://www.ncbi.nlm.nih.gov/projects/SNP/snp_ref.cgi?rs=1800795), IL6(rs1800797) means a single nucleotide polymorphism in the interleukin 6 (interferon, beta 2) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1800797).

“PHACTR2(rs7757372)” means a single nucleotide polymorphism in the phosphatase and actin regulator 2 (PHACTR2) gene. This is an A/G nucleotide substitution. The sequence surrounding this SNP is available from the dbSNP database of the National Center for Biotechnology www.ncbi.nlm.nih.gov/projects/SNP/snp_ref.cgi?rs=7757372), VDR(rs1544410) means a single nucleotide polymorphism in the vitamin D (1,25-dihydroxyvitamin D3) receptor (VDR) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1544410) and VDR(rs731236) means a single nucleotide polymorphism in the vitamin D (1,25-dihydroxyvitamin D3) receptor (VDR) gene. This is a C/T nucleotide substitution. The sequence surrounding this SNP is available from the dbSNP database of the National Center for Biotechnology (http://www.ncbi.nlm.nih.gov/projects/SNP/snp_ref.cgi?rs=731236).

“rs 1044122” means a single nucleotide polymorphism in the ADAM metallopeptidase domain 12 (ADAM12) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=1044122).

“rs10735810” means a single nucleotide polymorphism in the vitamin D (1,25-dihydroxyvitamin D3) receptor (VDR) gene. The sequence surrounding this SNP is available from the dbSNP database of the National Center for Biotechnology (www.ncbi.nlm.nih.gov/projects/SNP/snp_ref.cgi?rs=2228570).

“rs1143623” means a single nucleotide polymorphism in the interleukin 1, beta (IL1B) gene. This is a C/G nucleotide substitution. The sequence surrounding this SNP is available from the dbSNP database of the National Center for Biotechnology (www.ncbi.nlm.nih.gov/projects/SNP/snp_ref.cgi?rs=1143623).

“rs1143633” means a single nucleotide polymorphism in the interleukin 1, beta (IL1B) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1143633).

“rs1143634” means a single nucleotide polymorphism in the interleukin 1, beta (IL1B) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=1143634).

“rs1143643” means a single nucleotide polymorphism in the interleukin 1, beta (IL1B) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1143643).

“rs1165205” means a single nucleotide polymorphism in the interleukin 1, beta (IL1B) gene. This is an A/T nucleotide substitution. The sequence surrounding this SNP is available from the dbSNP database of the National Center for Biotechnology (www.ncbi.nlm.nih.gov/projects/SNP/snp_ref.cgi?rs=1165205).

“rs1278279” means a single nucleotide polymorphism in the ADAM metallopeptidase domain 12 (ADAM12) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1278279).

“rs12885300” means a single nucleotide polymorphism in the deiodinase, iodothyronine, type II (DIO2) gene. This is a C/T nucleotide substitution. The sequence surrounding this SNP is available from the dbSNP database of the National Center for Biotechnology (vvww.ncbi.nlm.nih.gov/projects/SNP/snp_ref.cgi?rs=12885300).

“rs1561888” means a single nucleotide polymorphism in the cartilage intermediate layer protein, nucleotide pyrophosphohydrolase (CILP) gene. This is an A/G nucleotide substitution. The sequence surrounding this SNP is available from the dbSNP database of the National Center for Biotechnology (www.ncbi.nlm.nih.gov/projects/SNP/snp_ref.cgi ?rs=1561888).

“rs1564858” means a single nucleotide polymorphism in the tumor necrosis factor receptor superfamily, member 11b (TNFRSF11B) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1564858).

“rs16890979” means a single nucleotide polymorphism in the solute carrier family 2 (facilitated glucose transporter), member 9 (SLC2A9) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=16890979).

“rs16944” means a single nucleotide polymorphism in the interleukin 1, beta (IL1B) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=16944).

“rs17561” means a single nucleotide polymorphism in the interleukin 1, alpha (IL1A) gene. This is a G/T nucleotide substitution. The sequence surrounding this SNP is available from the dbSNP database of the National Center for Biotechnology (www.ncbi.nlm.nih.gov/projects/SNP/snp_ref.cgi?rs=17561).

“rs 1800629” means a single nucleotide polymorphism in the tumor necrosis factor (TNF) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=1800629).

“rs1800796” means a single nucleotide polymorphism in the interleukin 6 (IL6) gene. This is a C/G nucleotide substitution. The sequence surrounding this SNP is available from the dbSNP database of the National Center for Biotechnology (www.ncbi.nlm.nih.gov/projects/SNP/snp_ref.cgi?rs=1800796).

“rs 1871054” means a single nucleotide polymorphism in the ADAM metallopeptidase domain 12 (ADAM12) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=1871054).

“rs2070739” means a single nucleotide polymorphism in the collagen, type II, alpha 1 (COL2A1) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=2070739).

“rs2073618” means a single nucleotide polymorphism in the tumor necrosis factor receptor superfamily, member 1 lb (TNFRSF11B) gene. This is a C/G nucleotide substitution. The sequence surrounding this SNP is available from the dbSNP database of the National Center for Biotechnology (www.ncbi.nlm.nih.gov/projects/SNP/snp_ref.cgi?rs=2073618).

“rs2073711” means a single nucleotide polymorphism in the cartilage intermediate layer protein, nucleotide pyrophosphohydrolase (CILP) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=2073711).

“rs225014” means a single nucleotide polymorphism in the deiodinase, iodothyronine, type II (DIO2) gene. The sequence surrounding this SNP is available from the dbSNP database of the National Center for Biotechnology (www.ncbi.nlm.nih.gov/projects/SNP/snp_ref.cgi?rs=225014).

“rs235768” means a single nucleotide polymorphism in the bone morphogenetic protein 2 (BMP2) gene. This is an A/T nucleotide substitution. The sequence surrounding this SNP is available from the dbSNP database of the National Center for Biotechnology (www.ncbi.nlm.nih.gov/projects/SNP/snp_ref.cgi?rs=235768).

“rs3181052” means a single nucleotide polymorphism in the interleukin 1 receptor antagonist (IL1RN) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=3181052).

“rs4720262” means a single nucleotide polymorphism in the thioredoxin domain containing 3 (TXNDC3) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=4720262).

“rs4848306” means a single nucleotide polymorphism in the IL1B gene with an A/G nucleotide substitution. The sequence surrounding this SNP is available from the dbSNP database of the National Center for Biotechnology (www.ncbi.nlm.nih.gov/projects/SNP/snp_ref.cgi?rs=4848306).

“rs4934” means a single nucleotide polymorphism in the serpin peptidase inhibitor, Glade A (alpha-1 antiproteinase, antitrypsin), member 3 (SERPINA3) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=4934).

“rs7172123” means a single nucleotide polymorphism in an unidentified gene on chromosome 15 with a C/T nucleotide substitution. The sequence surrounding this SNP is available from the dbSNP database of the National Center for Biotechnology (www.ncbi.nlm.nih.gov/projects/SNP/snp_ref.cgi?rs=7172123).

“rs751837” means a single nucleotide polymorphism in the CDC42 binding protein kinase beta (SERPINA3) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=751837).

“rs7628387” means a single nucleotide polymorphism in an unidentified gene on chromosome 3 with an A/C nucleotide substitution. The sequence surrounding this SNP is available from the dbSNP database of the National Center for Biotechnology (www.ncbi.nlm.nih.gov/projects/SNP/snp_ref.cgi?rs=7628387).

“rs7775” means a single nucleotide polymorphism in the frizzled-related protein (FRZB) gene. This is a C/G nucleotide substitution. The sequence surrounding this SNP is available from the dbSNP database of the National Center for Biotechnology (vvww.ncbi.nlm.nih.gov/projects/SNP/snp_ref.cgi?rs=7775).

rs9340799 means a single nucleotide polymorphism in the estrogen receptor 1 (ESR1) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=9340799).

Kellgren-Lawrence Grading Scale (“LK scale”) is used to measure occurrence and severity of osteoarthritis in human subjects. Grade 0 means the joints of a human subject is normal. Grade 1 means a human subject has doubtful narrowing of joint space and possible osteophytic lipping. Grade 2 means a human subject has definite osteophytes, definite narrowing of joint space. Grade 3 means a human subject has moderate multiple osteophytes, definite narrowing of joint space, some sclerosis and possible deformity of bone contour. Grade 4 means a human subject has large osteophytes, marked narrowing of joint space, severe sclerosis and definite deformity of bone contour.

The structural progression of OA is currently assessed on plain radiographic views by measuring the joint space width (JSW) and/or joint space narrowing (JSN) over a period of time. (Altman et al.: Osteoarthritis Cartilage 1996, 4:217-243.) OA progression is associated with accelerated cartilage degradation leading to joint space narrowing, painful joint disruption, and functional compromise. OA disease progression is measured on a LK scale.

Large amounts of data provide support for a central role of interleukin-1 (IL-1) in the pathogenesis of OA including animal susceptibility models, models of IL-1-targeted therapy, genetic association studies, and elevated IL-1 gene expression in whole blood from patients with generalized OA ((Loughlin et al., Arthritis Rheum 2002; 46(6):1519-27; Meulenbelt et al., Arthritis Rheum 2004; 50(4):1179-86; Moos et al., Arthritis Rheum 2000; 43(11):2417-22; Stern et al., Osteoarthritis Cartilage 2003; 11(6):394-402; Smith et al., Genes Immun 2004; 5(6):451-60; and Moxley et al., Osteoarthritis Cartilage 2007; 15(10):1106-12.). For example, evidence from the literature suggests that genetic predisposition is an important determinant of pathology in patients with hand OA (Moxley et al.: Osteoarthritis Cartilage 2007; 15(10):1106-12).

The term “allele” refers to the different sequence variants found at different polymorphic regions. For example, IL-1RN (VNTR) has at least five different alleles. The sequence variants may be single or multiple base changes, including without limitation insertions, deletions, or substitutions, or may be a variable number of sequence repeats.

The term “allelic pattern” refers to the identity of an allele or alleles at one or more polymorphic regions. For example, an allelic pattern may consist of a single allele at a polymorphic site, as for IL-1RN (VNTR) allele 1, which is an allelic pattern having at least one copy of IL-1 RN allele 1 at the VNTR of the IL-1 RN gene loci. Alternatively, an allelic pattern may consist of either a homozygous or heterozygous state at a single polymorphic site. For example, IL-1-RN (VNTR) allele 2,2 is an allelic pattern in which there are two copies of the second allele at the VNTR marker of IL-1RN that corresponds to the homozygous IL-RN (VNTR) allele 2 state. Alternatively, an allelic pattern may consist of the identity of alleles at more than one polymorphic site.

The term “control”, “control sample” or “reference” refers to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion of the IL-1 gene cluster. However, where the sample to be tested is genomic DNA, the control sample is preferably a highly purified sample of genomic DNA.

The term “haplotype” as used herein is intended to refer to a set of alleles that are inherited together as a group (are in linkage disequilibrium) at statistically significant levels (P_{corr}<0.05). As used herein, the phrase “an IL-1 haplotype” refers to a haplotype in the IL-1 loci. An IL-1 inflammatory or proinflammatory haplotype refers to a haplotype that is indicative of increased agonist and/or decreased antagonist activities.

The term “gene score” is calculated by counting the number of risk alleles or genotypes that an individual carries as a measure of their cumulative genetic risk. An example for that approach to calculating cumulative genetic risk is described in Zheng et al. (2008) “Cumulative Association of Five Genetic Variants with Prostate Cancer”, New England Journal of Medicine, Vol. 358, Pages 910-919. When such cumulative genetic risk may also be calculated to indicate a patient's future risk to, e.g., osteoarthristis progression, initiation and susceptibility. For example, a gene score of 2 or less indicates that such patient is at very low risk of osteoarthristis progression and a gene score of 3-4 indicates that the patient is at low risk of osteoarthristis progression. A gene score of 5-6 indicates that the patient is at risk of osteoarthristis progression while a gene score of 7 or above indicates that the patient is at high risk of osteoarthristis progression.

The terms “IL-1 gene cluster” and “IL-1 loci” as used herein include all the nucleic acid at or near the 2q13 region of chromosome 2, including at least the IL-1A, 1L-1B and 1L-1RN genes and any other linked sequences. (Nicklin et al., Genomics 19: 382-84, 1994). The terms “IL-1A”, “IL-1B”, and “IL-1RN” as used herein refer to the genes coding for IL-1, IL-1, and IL-1 receptor antagonist, respectively. The gene accession number for IL-1A, 1L-1B, and 1L-1RN are X03833, X04500, and X64532, respectively.

Genetic screening (also called genotyping or molecular screening), can be broadly defined as testing to determine if a patient has mutations (alleles or polymorphisms) that either cause a disease state or are “linked” to the mutation causing a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not'occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

As used herein, the term “OR” means odd ratio or the probability of osteoarthritis (“OA”) progression, initiation or susceptibility and is used to predict a patient's future risk of OA progression, initiation or susceptibility. For example, an OR of less than 0.25 indicates that the patient has a very low risk of OA progression, initiation or susceptibility. An OR of between 0.25 and 0.75 indicates that the patient has a low risk of OA progression, initiation or susceptibility. An OR above 1.75 indicates that the patient is at very high risk of OA progression, initiation or susceptibility and an OR of between 1.25 and 1.75 indicates that the patient has a high risk of OA progression, initiation or susceptibility. The term “Increased risk” refers to a statistically higher frequency of occurrence of the disease or condition in an individual carrying a particular polymorphic allele in comparison to the frequency of occurrence of the disease or condition in a member of a population that does not carry the particular polymorphic allele.

The term “interact” as used herein is meant to include detectable relationships or associations (e.g. biochemical interactions) between molecules, such as interactions between protein-protein, protein-nucleic acid, nucleic acid-nucleic acid and protein-small molecule or nucleic acid-small molecule in nature.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are 100% in linkage disequilibrium with the two most frequent alleles of IL-1 RN (VNTR), which are allele 1 and allele 2.

The term “marker” or “genetic marker” refers to a sequence in the genome that is known to vary among individuals. For example, the IL-1RN gene has a marker that consists of a variable number of tandem repeats (VNTR).

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele. A polymorphic region can be a single nucleotide, the identity of which differs in different alleles. A polymorphic region can also be several nucleotides long.

The term “OA susceptibility” means that certain alleles are hereby discovered to be associated with or predictive of a subject's incidence of developing osteoarthritis. The alleles are thus over-represented in frequency in individuals with OA as compared to healthy individuals. Thus, these alleles can be used to predict OA even in pre-symptomatic or pre-diseased individuals.

The term “OA progression” means that certain alleles are hereby discovered to be associated with or predictive of how fast a subject's osteoarthritis develops. The alleles are thus over-represented in frequency in individuals with fast OA development as compared to healthy individuals and to individuals with slower OA development. Thus, these alleles can be used to predict an OA patient's tendency to develop more severe form of OA.

The term “OA initiation” means that certain alleles are hereby discovered to be associated with or predictive of a subject's risk of developing osteoarthritis or a change from grades 0 and 1 to grade 2 and above on KL scale. The alleles are thus over-represented in frequency in individuals with high risk of developing OA as compared to healthy individuals. Thus, these alleles can be used to predict OA initiation even in pre-symptomatic or pre-diseased individuals.

The term “treating” as used herein is intended to encompass curing as well as ameliorating at least one symptom of a condition or disease.

The term “genotype or genotyping” means the combination of alleles that determines a specific trait of an individual or the particular alleles at specified loci present in an organism.

In one embodiment of the invention to predict OA progression in a patient, the biological sample is genotyped for (i) at least one of the genetic markers selected from the group consisting of BMP2 (rs 1049007), CLEC3B (rs 13963), IL1RN (rs 1374281), IL1RN (rs 1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961) and (ii) optionally one or more genetic markers selected from the group consisting of IL1RN (RS3181052), IL1RN (RS1794066), IL1RN (RS419598), IL1RN (RS9005), and IL1RN (RS315943)

In one embodiment of the invention to predict OA progression, the biological sample is genotyped for (i) at least one of the genetic markers selected from the group consisting of BMP2 (rs1049007), CLEC3B (rs13963), IL1RN (rs1374281), IL1RN (rs1794066), IL1RN (rs2637988), IL (rs315943), IL (rs315952), IL (rs380092), IL (rs4251961), IL1R1 (rs2287047), IL1R1 (rs315949), VDR (rs10735810), SLC17A3 (rs1165205), OPG (rs2073618), Cilp (rs2073711) and IL1RN (rs4251961) and (ii) IL1RN (rs419598) and IL1RN (rs9005). Preferably, the biological sample is genotyped for (i) IL1RN (rs315952) and (ii) IL1RN (rs419598) and IL1RN (rs9005); wherein a haplotype of rs419598/rs315952/rs9005 (TTA or TCG) indicates that said patient has low risk of osteoarthritis progression; wherein a haplotype of rs419598/rs315952/rs9005 (TTG) indicates that said patient has high risk of osteoarthritis progression. Alternatively, the biological sample is genotyped for IL1RN (rs419598), IL1RN (rs9005), and IL1RN (rs315943). A haplotype of rs419598/rs9005/rs315943 (AGT or AAT) indicates that said patient has low risk of osteoarthritis progression; and a haplotype of rs419598/rs315952/rs9005 (AGC) indicates that said patient has high risk of osteoarthritis progression.

In another embodiment of the invention, the biological sample is genotyped for at least two of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961). Preferably, the biological sample is genotyped for (i) IL1RN (rs315952) and IL1RN (rs315943) and (ii) IL1RN (rs579543) and IL1RN (rs9005); wherein a haplotype of rs579543/rs315952/rs9005/rs315943 (CCGT) (SEQ ID NO: 16) indicates that said patient has low risk of osteoarthritis progression; wherein a haplotype of rs579543/rs315952/rs9005/rs315943 (CTGC) (SEQ ID NO: 15) indicates that said patient has high risk of osteoarthritis progression. Preferably, rs419598 and rs315943 are genotyped.

In another embodiment of the invention, the biological sample is genotyped for at least three of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961). Preferably, the biological sample is genotyped for (i) IL1RN (rs4251961), IL1RN (rs419598) and IL1RN (rs315952) and (ii) IL1RN (rs9005); wherein a haplotype of rs4251961/rs419598/rs315952/rs9005 (TTCG) (SEQ ID NO: 17) indicates that said patient has low risk of osteoarthritis progression; wherein a haplotype of rs4251961/rs419598/rs315952/rs9005 (CTTG) (SEQ ID NO: 18) indicates that said patient has high risk of osteoarthritis progression. More preferably, the biological sample is genotyped for (i) IL (rs4251961), IL (rs2637988) and IL (rs1794066) and (ii) IL (rs3181052) and IL (rs419598); wherein a haplotype TAGAT (SEQ ID NO: 14) (rs4251961/rs2637988/rs3181052/rs1794066/rs419598) indicates that said patient has low risk of osteoarthritis progression; wherein a haplotype CAGAT (SEQ ID NO: 13) (rs4251961/rs2637988/rs3181052/rs1794066/rs419598) indicates that said patient has high risk of osteoarthritis progression. Preferably, rs419598, rs315943 and rs9005 are genotyped. More preferably rs419598, rs315943, rs315952 and rs9005 are genotyped and the most preferably rs419598, rs315943, rs315952, rs1794066 and rs9005 are genotyped.

Alternatively, biological sample is genotyped for IL1RN rs3181052|rs1794066|rs419598|RS9005|rs315943 wherein a haplotype GTAGT or GTAAT (rs3181052|rs1794066|rs419598|rs9005|rs315943) indicates that said patient has low risk of osteoarthritis progression; wherein a haplotype GTAGC (rs3181052|rs1794066|rs419598|rs9005|rs315943) indicates that said patient has high risk of osteoarthritis progression.

In another embodiment of the invention, the biological sample is genotyped for (i) at least six genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Culp (rs2073711) and IL1RN (rs4251961); and (ii) at least four of the genetic markers selected from the group consisting of IL1RN (rs419598), IL1RN (rs315931), IL1RN (rs3181052), IL1RN (rs579543) and IL1RN (rs9005). Preferably, rs3181052, rs1794066, rs419598, rs315952, rs9005 and rs315943 are genotyped.

Another embodiment of the invention includes further identification of an IL1RN haplotype which comprises at least seven markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943) and IL1RN(rs1374281); wherein said IL1RN haplotype with at least seven markers can be used to predict whether said patient is at high risk, neutral or low risk from OA progression. Preferably, the IL1RN haplotype comprises at least seven markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), IL1RN(rs1374281). More preferably, the IL1RN haplotype comprises at least ten markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), IL1RN(rs1374281), and more preferably, the IL haplotype comprises at least ten markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), IL1RN(rs1374281). Even more preferably, the haplotype of TCAGTAACTGCG (SEQ ID NO: 22)

(rs315931/rs4251961/rs2637988/rs3181052/rs1794066/rs419598/rs380092/rs579543/rs315952/r s9005/rs315943/rs1374281) indicate that said human subject is at risk of osteoarthritis progression; a haplotype of GTGGCGATTATC (SEQ ID NO: 1) (rs315931/rs4251961/rs2637988/rs3181052/rs1794066/rs419598/rs380092/rs579543/rs315952/r s9005/rs315943/rs1374281) indicates that said human subject is neutral to osteoarthritis progression; and a haplotype of TTAGTATCCGTC (SEQ ID NO: 2) (rs315931/rs4251961/rs2637988/rs3181052/rs1794066/rs419598/rs380092/rs579543/rs315952/r s9005/rs315943/rs1374281) indicates that said human subject is at low risk of osteoarthritis progression and the most preferably, the IL1RN haplotype comprises IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), IL1RN(rs1374281).

In another embodiment of the invention, the method of predicting progression of osteoarthritis further comprises the step of calculating gene score of said patient to predict severity of osteoarthritis progression, wherein a gene score of 2 or less indicates that such patient is at very low risk; wherein a gene score of 3-4 indicates that the patient is at low risk of osteoarthritis progression; wherein a gene score of 5-6 indicates that the patient is at risk of osteoarthritis progression; wherein a gene score of 7 or above indicates that the patient is at high risk of osteoarthritis progression. The biological sample can include, but not limited to saliva, buccal cells, blood, tissue samples or urine.

In one embodiment of the invention to predict OA initiation, biological sample is genotyped for at least two of the genetic markers selected from the group consisting of ADAM12 (rs3740199), BMP2 (rs1049007), CLEC3B (rs13963), HFE (rs1799945), IL1RN (rs315931), IL1RN (rs419598), IL1RN (rs579543), TURN (rs9005), IL1B(rs1143623), ADAM12(rs1871054), OPG(rs2073618), IL1RN(rs315943), IL1RN(rs315949), IL1RN(rs4251961), CDC42BPB(rs751837) and IL1RN(rs315952).

In another embodiment of the invention, the biological sample is genotyped for at least three of the genetic markers selected from the group consisting of ADAM12 (rs3740199), BMP2 (rs1049007), CLEC3B (rs13963), HFE (rs1799945), IL1RN (rs315931), IL1RN (rs419598), IL1RN (rs579543), IL1RN (rs9005), IL1B(rs1143623), ADAM12(rs1871054), OPG(rs2073618), IL1RN(rs315943), IL1RN(rs315949), IL1RN(rs4251961), CDC42BPB(rs751837) and IL1RN(rs315952). The biological sample is saliva, buccal cells, blood, tissue samples or urine. It can further comprise the step of calculating gene score of said patient to predict osteoarthritis initiation. Preferably at least four of the genetic markers are genotyped.

In another embodiment of the invention to predict OA initiation, gene scores are calculated to facilitate such prediction in patients, wherein a gene score of 2 or less indicates that such patient is at very low risk of osteoarthritis initiation; wherein a gene score of 3-4 indicates that the patient is at low risk of osteoarthritis initiation; wherein a gene score of 5-6 indicates that the patient is at risk of osteoarthritis initiation; wherein a gene score of 7 or above indicates that the patient is at high risk of osteoarthritis initiation.

In another embodiment of the invention to predict OA susceptibility, the biological sample is genotyped for at least two of the genetic markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236). Preferably, the biological sample is genotyped for at least three of the genetic markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236). More preferably, the biological sample is genotyped for at least four of the genetic markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236).

In another embodiment of the invention to predict a patient's susceptibility to osteoarthritis and/or initiation of osteoarthritis, a biological sample is taken from the patient and then genotyped for (i) at least one of the genetic markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236) and (ii) optionally IL1RN(rs315931), IL1RN (rs4251961), IL1RN (rs2637988), IL1RN (rs3181052), IL1RN (rs1794066), IL1RN (rs419598), IL1RN (rs380092), IL1RN (rs579543), IL1RN(rs9005), IL1RN(rs315943) and IL1RN(rs1374281); the genotyping results are compared with a reference; and prediction of susceptibility to osteoarthritis and/or initiation of osteoarthritis of the patient is based on the patient's genotype. Preferably, at least two of the genetic markers are genotyped and more preferably at least three or at least four of the genetic markers are genotyped. This embodiment can further comprise the step of identifying a haplotype comprising at least two markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236) and (ii) optionally IL1RN(rs315931), IL1RN (rs4251961), IL1RN (rs2637988), IL1RN (rs3181052), IL1RN (rs1794066), IL1RN.(rs419598), IL1RN (rs380092), IL1RN (rs579543), IL1RN(rs9005), IL1RN(rs315943) and IL1RN(rs1374281). Preferably the haplotype comprises VDR(1800797) and VDR(rs1800795). More preferably, it further comprises the step of calculating gene score of said patient to predict osteoarthritis susceptability. Most preferably a gene score of 2 or less indicates that such patient is at very low risk of osteoarthristis susceptibility; wherein a gene score of 3-4 indicates that the patient is at low risk of osteoarthristis susceptability; wherein a gene score of 5-6 indicates that the patient is at risk of osteoarthristis susceptibility; wherein a gene score of 7 or above indicates that the patient is at high risk of osteoarthristis susceptibility.

Another embodiment of the invention further comprises the step of identifying a haplotype comprising at least two markers selected from the group consisting of ABCG2(rs2231142), ADAM12(rs3740199), DVWA(rs11718863), ESR1(rs2234693), GDF5(rs143383), IL1A(rs10496444), IL1R1(rs2287047), IL6(rs1800795), IL6(rs1800797), PHACTR2(rs7757372), VDR(rs1544410) and VDR(rs731236) and (ii) optionally IL1RN(rs315931), IL1RN (rs4251961), IL1RN (rs2637988), IL1RN (rs3181052), IL1RN (rs1794066), IL1RN (rs419598), IL1RN (rs380092), IL1RN (rs579543), IL1RN(rs9005), IL1RN(rs315943) and IL1RN(rs1374281). Preferably the haplotype comprises VDR(1800797) and VDR(rs1800795).

In another embodiment of the invention, the prediction of OA susceptibility is accomplished by calculating gene score of said patient, wherein a gene score of 2 or less indicates that such patient is at very low risk of osteoarthritis susceptibility; wherein a gene score of 3-4 indicates that the patient is at low risk of osteoarthritis susceptibility; wherein a gene score of 5-6 indicates that the patient is at risk of osteoarthritis susceptibility; wherein a gene score of 7 or above indicates that the patient is at high risk of osteoarthritis susceptibility.

The term “comparing the genotyping results with a reference” means comparing genotyping results of the test individual with the control DNA samples of known sequences at the specified loci.

The term “multi-locus genotype” means the combination of alleles at multiple specific loci in the genome to explain biological behavior of the individual who provided the DNA.

The term “phenotype” means any observable characteristic or trait of an organism.

Haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). Oligonucleotides necessary for amplification may be selected, for example, from within the IL-1 gene loci, either flanking the marker of interest (as required for PCR amplification) or directly overlapping the marker (as in ASO hybridization). In a particularly preferred embodiment, the sample is hybridized with a set of primers, which hybridize 5′ and 3′ in a sense or antisense sequence to the vascular disease associated allele, and is subjected to a PCR amplification.

In a merely illustrative embodiment, the method includes the steps of (i) collecting a biological sample from a patient, (ii) isolating nucleic acid (e.g., genomic, mRNA or both) from the sample, (iii) contacting the nucleic acid sample with one or more primers which specifically hybridize 5′ and 3′ to at least one allele of an IL-1 proinflammatory haplotype under conditions such that hybridization and amplification of the allele occurs, and (iv) detecting the amplification product for the specific alleles that are of interest. These detection schemes are especially useful for the detection of nucleic acid molecules if such molecules are present in very low numbers.

The following examples are given as specific illustrations of the invention. It should be understood, however, that the invention is not limited to the specific details set forth in the examples. All parts and percentages in the examples, as well as in the remainder of the specification, are by weight unless otherwise specified.

Further, any range of numbers recited in the specification or paragraphs hereinafter describing or claiming various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited. The term “about” when used as a modifier for, or in conjunction with, a variable, is intended to convey that the numbers and ranges disclosed herein are flexible and that practice of the present invention by those skilled in the art using temperatures, concentrations, amounts, contents, carbon numbers, and properties that are outside of the range or different from a single value, will achieve the desired result.

### Example 1

### Genetic Markers Associated with Progression of OA

There are currently no approved drugs for the treatment or prevention of osteoarthritis (OA), due in part to the complexities of clinical trials in which only a small subset of patients show progression of the disease during the studies. Mechanisms underlying the progression of OA are not well understood. Although OA is not a classic inflammatory disease, inflammatory mediators that degrade cartilage have been implicated in its pathogenesis. We previously reported (Attur et al. 2009) that interleukin-1 receptor antagonist gene (IL1RN) variations (SNPs) were associated with knee OA severity. In the present study, Caucasian participants (N=1154; 38.2% men; mean age-60.3 years) in the Johnson County (JoCo) OA Project with 4-11 year follow-up data were selected to evaluate gene variations associated with radiographic knee OA progression.

Anterior-posterior standing knee radiographs were obtained with foot mat positioning at both time points and read by a single musculoskeletal radiologist for Kellgren Lawrence grade (K-L, 0-4). Median knee joint space narrowing (JSN) was also measured for both knees at the two time points.

Progression of knee OA was defined by an increase in KL grade or decrease in joint space width in at least one knee in subjects who already had OA (KL>=2 at either knee) at baseline. Genotypes of a broad panel of SNPs were obtained, including multiple genes and dense coverage of the IL-1 gene cluster (table 1). Logistic or linear regression with adjustment for age, gender and BMI was used to determine association between IL1RN gene polymorphisms and progression of knee OA.

TABLE 1
SNPs examined in the JoCo study

| SNP | Gene | Alleles |
|---|---|---|
| rs1044122 | ADAM12 | T/C |
| rs1049007 | BMP2 | A/G |
| rs10496444 | IL1A | C/T |
| rs10735810 | VDR | C/T |
| rs1143623 | IL1B | C/G |
| rs1143633 | IL1B | A/G |
| rs1143634 | IL1B | C/T |
| rs1143643 | IL1B | A/G |
| rs1165205 | SLC17A3 | A/T |
| rs11718863 | DVWA | A/T |
| rs1278279 | ADAM12 | A/G |
| rs12885300 | DIO2 | C/T |
| rs1374281 | IL1RN | C/G |
| rs13963 | CLEC3B | A/G |
| rs143383 | GDF5 | T/C |
| rs1544410 | VDR | A/G |
| rs1561888 | Clip | A/G |
| rs1564858 | OPG | A/G |
| rs16890979 | SLC2A9 | C/T |
| rs16944 | IL1B | A/G |
| rs17561 | IL1A | G/T |
| rs1794066 | IL1RN | A/G |
| rs1799945 | HFE | C/G |
| rs1800629 | TNFA | A/G |
| rs1800795 | IL6 | C/G |
| rs1800796 | IL6 | C/G |
| rs1800797 | IL6 | A/G |
| rs1871054 | ADAM12 | C/T |
| rs2070739 | COL2A1 | A/G |
| rs2073618 | OPG | C/G |
| rs2073711 | Cilp | C/T |
| rs2231142 | ABCG2 | A/C |
| rs2234693 | ESR1 | C/T |
| rs225014 | DIO2 | C/T |
| rs2287047 | IL1R1 | C/T |
| rs235768 | BMP2 | A/T |
| rs2637988 | IL1RN | A/G |
| rs315931 | IL1RN | A/C |
| rs315943 | IL1RN | C/T |
| rs315949 | IL1RN | C/T |
| rs315952 | IL1RN | C/T |
| rs3181052 | IL1RN | A/G |
| rs3740199 | ADAM12 | C/G |
| rs380092 | IL1RN | A/T |
| rs419598 | IL1RN | C/T |
| rs4251961 | IL1RN | C/T |
| rs4720262 | TXNDC3 | C/T |
| rs4848306 | IL1B | A/G |
| rs4934 | AACT | A/G |
| rs579543 | IL1RN | C/T |
| rs7172123 | Unidentified gene on | C/T |
| | chromosome 15 | |
| rs731236 | VDR | C/T |
| rs751837 | CDC42BPB | C/T |
| rs7628387 | Unidentified gene on | A/C |
| | chromosome 3 | |
| rs7757372 | PHACTR2 | A/G |
| rs7775 | FRZB | C/G |
| rs9005 | IL1RN | A/G |
| rs9340799 | ESR1 | A/G |

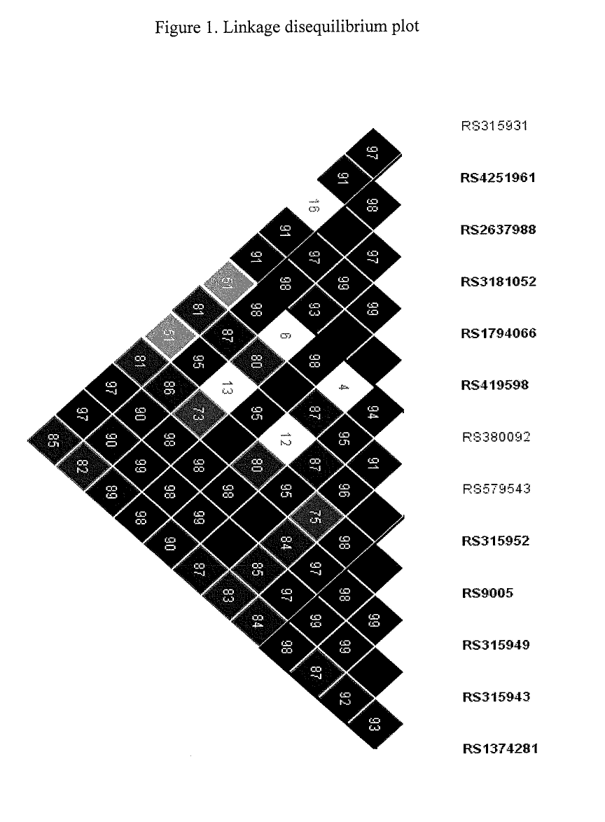

Specific SNPs and haplotypes of the BMP2, Cilp, CLEC3B, IL1RN, IL1R1, OPG, SLC17A3, VDR genes were significantly associated with progression of knee OA. There are 2 linkage disequilibrium (LD) blocks in the IL gene (FIG. 1), and markers in both blocks were significantly associated with progression of knee OA. Allele C of the IL1RN rs4251961, previously reported to be associated with reduced levels of the anti-inflammatory IL-1Ra protein, was associated with progression of knee OA (OR-3.07, 95% C1-1.50-6.28) (Table 2). Other SNPs that were associated with knee OA progression included rs1049007, rs13963, rs1374281, rs1794066, rs2637988, rs315943, rs315952, rs380092, rs2287047, and rs315949. rs10735810, rs1165205, rs2073618, rs2073711, (Tables 2 and 3).

TABLE 2
SNPs associated with progression of radiographic knee OA: change of KL score

| Gene | SNP | Allele | Model | n | OR | 95% CI(L) | 95% CI(U) | P |
|---|---|---|---|---|---|---|---|---|
| BMP2 | rs1049007 | G | REC | 124 | 2.37 | 1.03 | 5.45 | 0.0417 |
| CLEC3B | rs13963 | G | ADD | 121 | 1.86 | 1.10 | 3.15 | 0.0205 |
| IL1RN | rs1374281 | C | DOM | 154 | 2.97 | 1.48 | 5.97 | 0.0023 |
| IL1RN | rs1794066 | A | DOM | 153 | 3.60 | 1.40 | 9.22 | 0.0076 |
| IL1RN | rs2637988 | A | DOM | 153 | 2.98 | 1.18 | 7.55 | 0.0210 |
| IL1RN | rs315943 | C | DOM | 153 | 3.08 | 1.51 | 6.27 | 0.0019 |
| IL1RN | rs315952 | T | ADD | 153 | 1.78 | 1.05 | 3.00 | 0.0312 |
| IL1RN | rs380092 | A | ADD | 153 | 1.97 | 1.17 | 3.33 | 0.0109 |
| IL1RN | rs4251961 | C | DOM | 153 | 3.07 | 1.50 | 6.28 | 0.0021 |
| IL1R1 | rs2287047 | T | DOM | 150 | 2.45 | 1.21 | 4.98 | 0.0129 |
| IL1RN | rs315949 | T | DOM | 129 | 3.77 | 1.73 | 8.20 | 0.0008 |

SNP: single nucleotide polymorphism;
OR: odds ratio;
REC: recessive;
ADD: additive;
DOM: dominant;
n: number of samples;
95% CI(U): 95% confidence interval (upper);
95% CI(L): 95% confidence interval (lower);
p: probability

TABLE 3
SNPs associated with progression of
radiographic knee OA: change of JSW

| Gene | SNP | Allele | Model | n | Beta | P |
|---|---|---|---|---|---|---|
| BMP2 | rs1049007 | G | ADD | 70 | −0.06 | 0.033 |
| VDR | rs10735810 | C | ADD | 86 | −0.08 | 0.002 |
| SLC17A3 | Rs1165205 | T | DOM | 85 | −0.10 | 0.038 |
| OPG | Rs2073618 | G | ADD | 26 | −0.09 | 0.043 |
| Cilp | Rs2073711 | T | DOM | 74 | −0.09 | 0.040 |
| IL1RN | Rs4251961 | C | DOM | 87 | −0.08 | 0.041 |

SNP: single nucleotide polymorphism;
ADD: additive;
DOM: dominant;
n: number of samples;
Beta: regression coefficient;
p: probability

The IL1RN effect on risk for progression is attributable to several specific haplotypes composed of various numbers of IL1RN SNPs. For example Table 4a summarizes haplotypes composed of twelve IL1RN SNPs (rs315931/rs4251961/rs2637988/rs3181052/rs1794066/rs419598/rs380092/rs579543/rs315952/rs9005/rs315943/rs1374281) and their relationships with OA progression.

TABLE 4a
Frequency of IL1RN Haplotypes in OA Progressors and Non-Progressors

| | | SEQ | | | | |
|---|---|---|---|---|---|---|
| Disease | IL1RN | ID | Freq in | Freq in non- | | |
| Effect | HAPLOTYPE¹ | NO | Progressors² | Progressors | CHISQ | P |
| Neutral | GTGGCGATTA | 1 | 0.235 | 0.2437 | 0.0290 | 0.8647 |
| | TC | | | | | |
| Protective | TTAGTATCCG | 2 | 0.0841 | 0.1738 | 5.218 | 0.0224 |
| | TC | | | | | |
| Risk | TCAGTAACTG | 3 | 0.458 | 0.311 | 6.257 | 0.0124 |
| | CG | | | | | |

¹IL1RN SNPs: rs315931/rs4251961/rs2637988/rs3181052/rs1794066/rs419598/
rs380092/rs579543/rs315952/rs9005/rs315943/rs1374281
²Frequency of the haplotype in knee OA cases (Kellgren-Lawrence scores
of ≧2) that exhibit an increase in KL score during a 4 to 11 year
follow-up.

One haplotype (GTGGCGATTATC) is basically neutral and is not differentially represented in either progressors or non-progressors. One haplotype (TTAGTATCCGTC) is protective and is more than twice as frequent in non-progressors compare to progressors. The third haplotype (TCAGTAACTGCG) is associated with increased risk for progression.

TABLE 4b
IL1RN Haplotype (with 12 SNPs) -
Risk for OA Progression

| | | SEQ | | |
|---|---|---|---|---|
| Disease | IL1RN | ID | | |
| Effect | HAPLOTYPE¹ | NO | OR² | P |
| Neutral | GTGGCGATTATC | 4 | 0.93 | 0.798 |
| Protective | TTAGTATCCGTC | 5 | 0.40 | 0.0281 |
| Risk | TCAGTAACTGCG | 6 | 1.96 | 0.0094 |

¹IL1RNSNPs: rs315931/rs4251961/rs2637988/
rs3181052/rs1794066/rs419598/rs380092/
rs579543/rs315952/rs9005/rs315943/rs1374281
²Odds ratio for the indicated haplotype being
associated with knee OA cases (Kellgren-Lawrence
scores of ≧2) that exhibit an increase in KL
score during a 4 to 11 year follow-up after
adjustment for age, BMI, and gender.

The specific 12 SNPs were selected to capture the majority of the variation in the IL1RN gene. Other SNPs can be selected that tag the 3 critical haplotypes identified in Table 4a and 4b. Therefore any combinations of SNPs that tag these key haplotypes are in fact merely identifying the same haplotypes. That is clearly demonstrated by Table 4e, in which we show that multiple subsets of the 12 SNPs may be used to tag the critical extended IL1RN haplotypes. For example, several IL1RN haplotypes, as shown in Table 4c, including the IL1RN (rs419598/315952/9005) TTG haplotype previously shown to be associated with severity of knee OA in the NYU/Duke studies, were associated with progression of disease in this cohort study. There were also haplotyes not associated either increased or decreased risk for OA progression.

TABLE 4c
IL1RN Haplotype (with 13 SNPs) -
Risk for OA Progression

| | | SEQ | | |
|---|---|---|---|---|
| Disease | IL1RN | ID | | |
| Effect | HAPLOTYPE¹ | NO | OR² | P |
| Neutral | CTGGGCATTACTG | 7 | 0.93 | 0.798 |
| Protective | ATAGATTCCGCTG | 8 | 0.40 | 0.028 |
| Risk | ACAGATACTGTCC | 9 | 1.96 | 0.009 |

¹IL1RNSNPs: rs315931/rs4251961/rs2637988/
rs3181052/rs1794066/rs419598/rs380092/
rs579543/rs315952/rs9005/rs315949/
rs315943/rs1374281
²Odds ratio for the indicated haplotype being
associated with knee OA cases (Kellgren-Lawrence
scores of ≧2) that exhibit an increase in KL
score during a 4 to 11 year follow-up after
adjustment for age, BMI, and gender.

TABLE 4d
IL1RN Haplotype (with 10 SNPs) -
Risk for OA Progression

| | | SEQ | | |
|---|---|---|---|---|
| Disease | IL1RN | ID | | |
| Effect | HAPLOTYPE₁ | NO | OR₂ | P |
| Neutral | CTCATTACTG | 10 | 0.90 | 0.726 |
| Protective | ATTTCCGCTG | 11 | 0.46 | 0.009 |
| Risk | ACTACTGTCC | 12 | 1.96 | 0.010 |

₁IL1RN SNPs:rs315931/rs4251961/rs419598/
rs380092/rs579543/rs315952/rs9005/rs315949/
rs315943/rs1374281
₂Odds ratio for the indicated haplotype being
associated with knee OA cases (Kellgren- Lawrence
scores of ≧2) that exhibit an increase in KL
score during a 4 to 11 year follow-up after
adjustment for age, BMI, and gender.

TABLE 4e
Haplotypes associated with progression of radiographic knee OA: change of KL score

| | | | SEQ | | | |
|---|---|---|---|---|---|---|
| Gene | SNPs | Haplotype | ID NO | Frequency | OR | P |
| IL1RN | rs4251961/rs2637988/rs3181052/ | CAGAT | 13 | 0.38 | 1.80 | 0.020 |
| | rs1794066/rs419598 | | | | | |
| IL1RN | rs4251961/rs2637988/rs3181052/ | TAGAT | 14 | 0.22 | 0.52 | 0.042 |
| | rs1794066/rs419598 | | | | | |
| IL1RN | rs579543/rs315952/rs9005/ | CTGC | 15 | 0.41 | 1.97 | 0.008 |
| | rs315943 | | | | | |
| IL1RN | rs579543/rs315952/rs9005/ | CCGT | 16 | 0.28 | 0.55 | 0.024 |
| | rs315943 | | | | | |
| IL1RN | rs4251961/rs419598/rs315952/ | TTCG | 17 | 0.27 | 0.58 | 0.040 |
| | rs9005 | | | | | |
| IL1RN | rs4251961/rs419598/rs315952/ | CTTG | 18 | 0.36 | 1.95 | 0.011 |
| | rs9005 | | | | | |
| IL1RN | rs419598/rs315952/rs9005 | TTA | | 0.04 | 0.09 | 0.024 |
| IL1RN | rs419598/rs315952/rs9005 | TCG | | 0.28 | 0.59 | 0.044 |
| IL1RN | rs419598/rs315952/rs9005 | TTG | | 0.42 | 1.97 | 0.008 |

SNP: single nucleotide polymorphism; OR: odds ratio; p: probability

Multi-Locus Genotypes are Associated with Knee OA Progression

To assess the combined effect of multiple gene variants on progression of OA, polygenic risk models were developed using SNPs associated with OA progression. These variants include rs1049007 (BMP2), rs13963 (CLEC3B), rs4251961 (ILl RN), rs2287047 (IL1R1) and rs315949. More than 20 composite genotype patterns were identified (Table 5a). These patterns were associated with various levels of risk for OA progression, ranging from highly protective (RR=0.15) to highly risk (RR=2.89).

TABLE 5a
Composite genotype patterns associated with risk for OA progression:
polygenic risk model

| | Genotype | | | | | Relative | |
|---|---|---|---|---|---|---|---|
| Pattern | rs1049007 | rs13963 | rs4251961 | rs2287047 | rs315949 | risk¹ | Freq |
| Highly | A/* | GG | TT | CC | CC | 0.15 | 0.47 |
| protective | A/* | GG | TT | T/* | CC | | |
| | A/* | AG | TT | CC | CC | | |
| | A/* | AG | TT | T/* | CC | | |
| | A/* | AA | C/* | CC | CC | | |
| | A/* | AA | TT | CC | CC | | |
| | A/* | AA | TT | T/* | CC | | |
| | A/* | AA | TT | CC | T/* | | |
| | GG | GG | TT | CC | CC | | |
| Protective | A/* | GG | TT | CC | T/* | 0.66 | 0.19 |
| | A/* | AG | C/* | CC | T/* | | |
| | A/* | AG | C/* | T/* | CC | | |
| | A/* | AA | C/* | CC | T/* | | |
| | GG | GG | TT | T/* | CC | | |
| | GG | AA | C/* | CC | T/* | | |
| Risk | A/* | AG | C/* | CC | T/* | 1.32 | 0.11 |
| | GG | AG | C/* | CC | T/* | | |
| Highly | A/* | GG | C/* | CC | T/* | 2.89 | 0.23 |
| risk | A/* | GG | C/* | T/* | T/* | | |
| | A/* | AG | C/* | T/* | T/* | | |
| | A/* | AA | C/* | T/* | T/* | | |
| | GG | GG | C/* | CC | T/* | | |
| | GG | AG | C/* | T/* | T/* | | |
| | GG | AA | C/* | T/* | T/* | | |

Table 5b shows the same data represented as a “gene-score” in which the risk alleles are counted and the risk is stratified based on the number of risk alleles. This example includes 5 SNPs but can include all of the risk alleles identified in the study, including the IL1RN haplotypes as one set of risk alleles.

TABLE 5b
Gene-Score patterns associated with risk for OA progression

| | Genotype | | | | | | | | | | Gene |
|---|---|---|---|---|---|---|---|---|---|---|---|
| Pattern | rs1049007 | | rs13963 | | rs4251961 | | rs2287047 | | rs315949 | | Score |
| Very low | A/* | 0 | AA | 0 | TT | 0 | CC | 0 | CC | 0 | 0 |
| risk | A/* | 0 | AG | 1 | TT | 0 | CC | 0 | CC | 0 | 1 |
| | A/* | 0 | AA | 0 | C/* | 2 | CC | 0 | CC | 0 | 2 |
| | A/* | 0 | GG | 2 | TT | 0 | CC | 0 | CC | 0 | 2 |
| | A/* | 0 | AA | 0 | TT | 0 | T/* | 2 | CC | 0 | 2 |
| | A/* | 0 | AA | 0 | TT | 0 | CC | 0 | T/* | 2 | 2 |
| Low risk | A/* | 0 | AG | 1 | TT | 0 | T/* | 2 | CC | 0 | 3 |
| | A/* | 0 | GG | 2 | TT | 0 | T/* | 2 | CC | 0 | 4 |
| | GG | 2 | GG | 2 | TT | 0 | CC | 0 | CC | 0 | 4 |
| | A/* | 0 | GG | 2 | TT | 0 | CC | 0 | T/* | 2 | 4 |
| | A/* | 0 | AA | 0 | C/* | 2 | CC | 0 | T/* | 2 | 4 |
| Risk | A/* | 0 | AG | 1 | C/* | 2 | CC | 0 | T/* | 2 | 5 |
| | A/* | 0 | AG | 1 | C/* | 2 | T/* | 2 | CC | 0 | 5 |
| | A/* | 0 | AG | 1 | C/* | 2 | CC | 0 | T/* | 2 | 5 |
| | GG | 2 | GG | 2 | TT | 0 | T/* | 2 | CC | 0 | 6 |
| | GG | 2 | AA | 0 | C/* | 2 | CC | 0 | T/* | 2 | 6 |
| | A/* | 0 | GG | 2 | C/* | 2 | CC | 0 | T/* | 2 | 6 |
| | A/* | 0 | AA | 0 | C/* | 2 | T/* | 2 | T/* | 2 | 6 |
| High risk | GG | 2 | AG | 1 | C/* | 2 | CC | 0 | T/* | 2 | 7 |
| | A/* | 0 | AG | 1 | C/* | 2 | T/* | 2 | T/* | 2 | 7 |
| | A/* | 0 | GG | 2 | C/* | 2 | T/* | 2 | T/* | 2 | 8 |
| | GG | 2 | AA | 0 | C/* | 2 | T/* | 2 | T/* | 2 | 8 |
| | GG | 2 | GG | 2 | C/* | 2 | CC | 0 | T/* | 2 | 8 |
| | GG | 2 | AG | 1 | C/* | 2 | T/* | 2 | T/* | 2 | 9 |

These findings validate previous observations pointing to a genetic contribution of the IL1RN gene to knee OA progression and severity. This information could assist in guiding clinical development of new drugs for OA.

### Example 2

### Genetic Markers Associated with Initiation of OA

Caucasian participants (N=1154; 38.2% men; mean age=60.3 years) in the Johnson County (JoCo) OA Project with 4-11 year follow-up data were selected to evaluate gene variations associated with radiographic knee OA initiation. Anterior-posterior standing knee radiographs were obtained with foot mat positioning at both time points and read by a single musculoskeletal radiologist for Kellgren-Lawrence grade (K-L, 0-4). Median knee joint space width (JSW) was also measured for both knees at the two time points.

Initiation of knee OA was defined by an increase in KL grade or decrease in JSW in at least one knee in subjects without OA (KL<=1 at both knees) at baseline. Genotypes of a broad panel of SNPs were obtained, including multiple genes and dense coverage of the IL-1 gene cluster (table 1). Logistic or linear regression with adjustment for age, gender and BMI was used to determine association between IL1RN gene polymorphisms and initiation of knee OA.

TABLE 6
SNPs associated with initiation of
radiographic knee OA: change of KL score

| | | | | | | 95% | 95% |
|---|---|---|---|---|---|---|---|
| Gene | SNP | Allele | Model | n | OR | CI(L) | CI(U) |
| ADAM12 | rs3740199 | G | DOM | 893 | 1.47 | 1.02 | 2.12 |
| BMP2 | rs1049007 | G | REC | 719 | 1.38 | 1.01 | 1.90 |
| CLEC3B | rs13963 | G | ADD | 707 | 1.29 | 1.01 | 1.63 |
| HFE | rs1799945 | G | ADD | 866 | 1.32 | 1.01 | 1.73 |
| IL1RN | rs315931 | G | REC | 905 | 1.83 | 1.12 | 2.98 |
| IL1RN | rs419598 | G | REC | 903 | 2.00 | 1.14 | 3.50 |
| IL1RN | rs579543 | T | REC | 901 | 2.15 | 1.26 | 3.66 |
| IL1RN | rs9005 | A | REC | 905 | 2.17 | 1.33 | 3.54 |

SNP: single nucleotide polymorphism;
OR: odds ratio;
REC: recessive;
ADD: additive;
DOM: dominant;
n: number of samples;
95% CI(U): 95% confidence interval (upper);
95% CI(L): 95% confidence interval (lower);
p: probability

TABLE 7
SNPs associated with initiation of
radiographic knee OA: change of JSW

| Gene | SNP | Allele | Model | n | Beta | P |
|---|---|---|---|---|---|---|
| IL1B | rs1143623 | G | REC | 347 | −0.08 | 0.042 |
| ADAM12 | rs1871054 | T | REC | 347 | −0.07 | 0.042 |
| OPG | rs2073618 | G | REC | 97 | −0.09 | 0.043 |
| IL1RN | rs315943 | C | REC | 345 | −0.07 | 0.005 |
| IL1RN | rs315949 | T | REC | 295 | −0.07 | 0.010 |
| IL1RN | rs4251961 | C | REC | 345 | −0.07 | 0.005 |
| CDC42BPB | rs751837 | C | REC | 339 | −0.29 | 0.016 |

SNP: single nucleotide polymorphism;
REC: recessive;
n: number of samples;
Beta: regression coefficient;
p: probability

TABLE 8
IL1RN haplotypes associated with initiation of radiographic
knee OA: change of JSW

| SNPs | Haplotype | Frequency | Beta | P |
|---|---|---|---|---|
| rs419598/rs315952/rs9005 | CTA | 0.26 | 0.04 | 0.043 |
| rs419598/rs315952/rs9005 | TTA | 0.04 | 0.04 | 0.446 |
| rs419598/rs315952/rs9005 | TCG | 0.28 | 0.02 | 0.428 |
| rs419598/rs315952/rs9005 | TTG | 0.42 | −0.05 | 0.003 |

SNP: single nucleotide polymorphism;
Beta: regression coefficient;
p: probability
Note:
for this analysis, only subjects with KL = 0 at baseline were included.

Specific SNPs and haplotypes of the ADAM12, BMP2, CDC42BPB, CLEC3B, HFE, IL1B, 1L1RN and OPG genes were significantly associated with initiation of knee OA (Tables 6 and 7). There are 2 LD blocks in the IL1RN gene, and markers in both blocks were significantly associated with initiation of knee OA. Allele C of the IL1RN rs4251961, previously reported to be associated with reduced levels of the anti-inflammatory IL-1 Ra protein, was associated with initiation of knee OA (linear regression, p=0.005). Other SNPs that were associated with knee OA initiation included rs3740199, rs1049007, rs13963, rs1799945, rs315931, rs419598, rs579543, rs9005, rs1143623, rs1871054, rs2073618, rs315943, rs315949, and rs751837. The haplotype effect of the 2^{nd} block (block #7) is captured primarily by a single SNP (rs315943) (Table 9). The IL1RN (rs419598/315952/9005) TTG haplotype, previously shown to be associated with severity of knee OA, was associated with initiation of disease in this cohort study (Table 8).

TABLE 9
Conditional haplotype analysis: IL1RN block #6

| Haplotype block | | | | | |
|---|---|---|---|---|---|
| rs579543/rs315952/ | | Controlled SNP | | | |
| rs9005/rs315943 | | rs579543 | rs315952 | rs9005 | rs315943 |
| p value | 0.008 | 0.007 | 0.021 | 0.010 | 0.789 |

### Example 3

### Genetic Markers Associated with Susceptibility to OA

Factors that differentiate individuals who develop osteoarthritis (OA) from those who do not may be valuable in developing prevention strategies. Although several genetic variants have been associated with susceptibility to OA, most have not been replicated in adequately sized cohorts. We therefore sought to validate genetic variants predictive of OA susceptibility in a Caucasian patient sample in the United States, in a population-based study. Caucasian participants (N=1154; 38.2% men; mean age=60.3 years) in the Johnson County (JoCo) OA Project with 4-11 year follow-up data were examined. To identify markers associated with susceptibility to radiographic knee OA, a cross-sectional analysis was performed using data from follow up (T1) time point. Anterior-posterior standing knee radiographs were obtained with foot mat positioning at T1 time point and read by a single musculoskeletal radiologist for Kellgren-Lawrence grade (K-L, 0-4). OA cases were defined as having KL>=2 in at least one knee. Non-OA controls were defined as having KL=0 in both knees. Genotypes of 58 single nucleotide polymorphisms (SNPs) in 26 genes, including gene variants previously shown to be associated with OA and variants in genes that are functionally implicated in OA, such as the proinflammatory IL-1 gene family, were determined using the single-nucleotide primer extension method. Logistic regression with adjustment for age, gender and body mass index was used to determine associations between gene polymorphisms and susceptibility to radiographic knee OA. An association was considered a positive validation if the p-value after adjustment for age, gender and BMI <0.05 for the risk allele, genotype or haplotype previously reported to be associated with OA. Out of 26 genes tested, 10 were significantly associated with susceptibility to radiographic knee OA. These included ABCG2, ADAM12, DVWA, ESR1, GDF5, ILIA, IL1R1, IL6, PHACTR2 and VDR genes (Table 10). In addition, several haplotypes in the IL1RN or VDR gene were associated with susceptibility to knee OA (Table 11).

TABLE 10
Genetic variants associated with susceptibility to radiographic knee OA

| Gene | SNP | Allele | Model | n | OR | 95% CI(L) | 95% CI(U) | P |
|---|---|---|---|---|---|---|---|---|
| ABCG2 | rs2231142 | C | REC | 717 | 1.56 | 1.03 | 2.38 | 0.038 |
| ADAM12 | rs3740199 | G | DOM | 734 | 1.58 | 1.03 | 2.44 | 0.038 |
| DVW A | rs11718863 | A | ADD | 718 | 1.42 | 1.03 | 1.95 | 0.031 |
| ESR1 | rs2234693 | C | ADD | 607 | 1.32 | 1.02 | 1.71 | 0.035 |
| GDF5 | rs143383 | T | DOM | 742 | 1.66 | 1.04 | 2.65 | 0.034 |
| IL1A | rs10496444 | C | REC | 746 | 1.81 | 1.04 | 3.15 | 0.035 |
| ILR1 | rs2287047 | C | DOM | 729 | 2.06 | 1.02 | 4.15 | 0.043 |
| IL6 | rs1800795 | C | REC | 703 | 1.72 | 1.11 | 2.66 | 0.015 |
| IL6 | rs1800797 | A | REC | 749 | 1.83 | 1.19 | 2.82 | 0.006 |
| PHACTR2 | rs7757372 | A | REC | 515 | 1.55 | 1.04 | 2.31 | 0.032 |
| VDR | rs1544410 | A | DOM | 746 | 1.44 | 1.02 | 2.04 | 0.040 |
| VDR | rs731236 | C | DOM | 745 | 1.57 | 1.11 | 2.23 | 0.011 |

SNP: single nucleotide polymorphism;
OR: odds ratio;
REC: recessive;
ADD: additive;
DOM: dominant;
n: number of samples;
95% CI(U): 95% confidence interval (upper);
95% CI(L): 95% confidence interval (lower);
p: probability

This study validated several genetic markers for association with susceptibility to radiographic knee OA in a population-based study of Caucasians.

TABLE 11
Haplotyes associated with susceptibility to radiographic knee OA

| Gene | SNPs | Haplotype | Frequency | OR | P |
|---|---|---|---|---|---|
| IL1RN | rs315931/rs4251961/rs2637988/ | ATAGAT | 0.38 | 1.80 | 0.020 |
| | rs3181052/rs1794066/rs419598/ | TCCGTG | | | |
| | rs380092/rs579543/rs315952/ | (SEQ ID | | | |
| | rs9005/rs315943/rs1374281 | NO: 19) | | | |
| IL1RN | rs4251961/rs2637988/rs3181052/ | TAGAT | 0.22 | 0.74 | 0.04 |
| | rs1794066/rs419598 | (SEQ ID | | | |
| | | NO: 20) | | | |
| VDR | rs1800797/rs1800795 | AC | 0.44 | 1.32 | 0.03 |
| VDR | rs1800797/rs1800795 | GG | 0.55 | 0.73 | 0.01 |

SNP: single nucleotide polymorphism; OR: odds ratio; p: probability.

### Example 4

### Radiographic Kellgren-Lawrence (KL) grade 1 is Genetically Distinct from KL 0: Implications for Genetic Studies of Knee Osteoarthritis (OA)

The Kellgren-Lawrence (KL) radiographic grading system is widely used in studies of osteoarthritis (OA). Although KL grades 1 and 0 together often form the control group in epidemiologic studies, Hart and Spector (2003) showed different knee OA progression rates for KL1 and KLO, suggesting distinct phenotypes. We explored whether KL grades 1 and 0 are genetically distinct by comparing frequencies of genetic markers between subjects with the two KL grades.

Caucasian participants (N=1154; 38.2% men; mean age=60.3 years) in the Johnson County (JoCo) OA Project with 4-11 year follow-up data were examined. Anterior-posterior standing knee radiographs were obtained with foot mat positioning at both time points and read by a single musculoskeletal radiologist for K-L grades (0-4). Genotypes of 58 single nucleotide polymorphisms (SNPs) in 26 genes reported to be associated with OA were determined using the single-nucleotide primer extension method. Incidence of OA was defined by an increase in KL grade at follow up, in those with KL 0 bilaterally at baseline. Differences in genotype or allele frequencies between KL1 and KLO and between subjects with incident OA and those without incident OA were determined by Chi-Square test or logistic regression with adjustment for age, gender and body mass index (BMI). An association was considered positive if the adjusted p-value was <0.05 for the risk allele or genotype.

TABLE 12
Distribution of genetic markers between KL0 and KL1

| | | Allele/ | | Frequency | | |
|---|---|---|---|---|---|---|
| Gene | SNP | Genotype | n | KL = 1 | KL = 0 | P |
| ABCG2 | rs2231142 | A/A, A/C | 745 | 0.17 | 0.23 | 0.049 |
| ADAM12 | rs3740199 | C/C, C/G | 760 | 0.62 | 0.69 | 0.036 |
| DVWA | rs11718863 | T/T, T/A | 743 | 0.27 | 0.36 | 0.007 |
| IL1RN | rs419598 | C/C | 770 | 0.08 | 0.04 | 0.012 |
| IL1RN | rs579543 | T/T | 767 | 0.09 | 0.04 | 0.003 |
| IL1RN | rs9005 | A/A | 771 | 0.10 | 0.06 | 0.017 |
| IL6 | rs1800797 | A | 773 | 0.47 | 0.41 | 0.012 |
| PHACTR2 | rs7757372 | G/G, G/A | 518 | 0.35 | 0.46 | 0.020 |

SNP: single nucleotide polymorphism;
n: number of samples;
KL: Kellgren-Lawrence score;
p: probability

Compared to subjects with KLO (n=396), those with KL1 (n=381) were older (65.4 yrs vs. 62.9 yrs) and heavier (BMI 29.2 kg/m² vs. 28.3). Frequencies of alleles or genotypes in 6 genes, including ABCG2, ADAM12, DVWA, IL1RN, IL6, and PHACTR2, were significantly different between KL0 and KL1 subjects (Table 12). Among these genetic markers, six variants in 3 genes, IL1RN (rs419598, p=0.017; rs579543, p=0.003; and rs9005, v0.005), IL6 (rs1800795, p=0.049 and rs1800797, p=0.021) and PHACTR2 (rs7757372, p=0.036), were also associated with incidence of radiographic knee OA (Table 13). In addition, compared to KLO subjects, KL1 subjects were more likely to progress to KL>=2 (33.24% vs 8.45%) (Table 14) as previously reported. No population genetic substructure was detected in this Caucasian population.

TABLE 13
SNPs associated with incidence of knee OA

| Gene | SNP | Allele | Model | n | OR | 95% CI(L) | 95% CI(U) | P |
|---|---|---|---|---|---|---|---|---|
| IL1RN | rs419598 | C | REC | 548 | 2.60 | 1.18 | 5.71 | 0.017 |
| IL1RN | rs579543 | T | REC | 547 | 3.05 | 1.45 | 6.42 | 0.0030 |
| IL1RN | rs9005 | A | REC | 551 | 2.62 | 1.34 | 5.11 | 0.005 |
| IL6 | rs1800795 | C | ADD | 522 | 1.30 | 1.00 | 1.69 | 0.049 |
| IL6 | rs1800797 | A | ADD | 553 | 1.35 | 1.05 | 1.75 | 0.021 |
| PHACTR2 | rs7757372 | A | ADD | 371 | 1.47 | 1.03 | 2.12 | 0.036 |

SNP: single nucleotide polymorphism;
OR: odds ratio;
REC: recessive;
ADD: additive;
n: number of samples;
95% CI(U): 95% confidence interval (upper);
95% CI(L): 95% confidence interval (lower);
p: probability

TABLE 14
Frequencies of follow up KL grades between subjects with
grade 0 and grade 1 at baseline

| | | Follow Up | | | |
|---|---|---|---|---|---|
| | n | KL = 0 | KL = 1 | KL ≧ 1 | KL = 2 |
| KL = 0 at baseline | 556 | 63.85 | 27.70 | 36.15 | 8.45 |
| KL = 1 at baseline | 361 | 6.93 | 59.83 | 93.07 | 33.24 |

n: number of samples;
KL: Kellgren-Lawrence score

This study provides genetic evidence to support differentiating KL1 and KLO subjects in radiographic knee OA studies.

Haplotypes were generated for 12 SNPs assayed (two of the 13 assayed were in perfect linkage disequilibrium, so only one was included in the models) in the IL1RN gene. We then used backwards elimination modeling (Francis PLoS One 2007) to determine the best set of IL1RN markers that captured the influence of the variations in that gene on radiographic progression of knee OA in this population. For backwards elimination, the first model included 12 SNPs, and then one SNP was removed at a time producing models, each with 11 SNPs. The model with the lowest overall p-value was selected as the next model. This process was repeated to produce the best models for each number of SNPs. We used the Bonferroni adjusted p-value to account for multiple testing. For each model for a given number of SNPs, we used haplo.stat to estimate haplotype frequencies for cases and controls and to estimate an odds ratio for each individual haplotype to determine if individual haplotypes differed significantly between cases and controls. Based on the Omnibus overall p-values, the models with 3 to 5 SNPs were the strongest (Table 15a).

Table 15b shows the frequencies of the best 3, 4, and 5-SNP haplotypes in knee OA progressors and non-progressors. The 3-SNP model including RS419598|RS9005|RS315943 appears to be optimal because there are haplotypes with substantial frequencies that are significant predictors of increased risk (AGC; p=0.005); decreased risk (AGT; p=0.03); and with no observable influence on risk (GAT; p=0.60). We conclude that the IL1RN haplotypes identified are good predictors of radiographic progression and may be tagged by various combinations of SNPs, such as those shown in our models.

TABLE 15a

| | | SEQ | | | | |
|---|---|---|---|---|---|---|
| | | ID | | | | P-value |
| SNPs | HAPLOTYPE | NO | #SNPs | Freq | OR | ADJUSTED |
| RS315931\|RS4251961\|RS2637988\| | GTGGCGATTAT | 21 | 12 | 0.236 | 0.926 | 0.798 |
| RS3181052IRS1794066\|RS419598\| | C | | | | | |
| RS380092\|RS579543\|RS315952\| | TCAGTAACTGC | 22 | | 0.344 | 1.96 | 0.00944 |
| RS9005\|RS315943\|RS137428 | G | | | | | |
| | TTAGTAACTGC | 23 | | 0.0271 | 1.15 | 0.88 |
| | G | | | | | |
| | TTAGTATCCGT | 24 | | 0.142 | 0.401 | 0.0281 |
| | C | | | | | |
| | TTGACATCCGT | 25 | | 0.0894 | 0.606 | 0.165 |
| | C | | | | | |
| | GTGACATCCGT | 26 | | 0.0261 | 1.34 | 0.622 |
| | C | | | | | |
| | GTGGCGATTAT | 27 | | 0.0103 | 1.07E+09 | 0.999 |
| | G | | | | | |
| | TCAGTAATTAT | 28 | | 0.0111 | 5.42E−10 | 0.999 |
| | C | | | | | |
| | TTAGTATCTGC | 29 | | 0.0187 | 1.36E+09 | 0.999 |
| | G | | | | | |
| | TCAGTAACTGC | 30 | | 0.0172 | 0.661 | 0.689 |
| | C | | | | | |
| | TTAGCGATTAT | 31 | | 0.0114 | 2.25 | 0.349 |
| | C | | | | | |
| Omnibus model p-value | 0.05243 | | | | | |
| RS315931\|RS4251961\|RS2637988\| | GTGGCGATATC | 32 | 11 | 0.232 | 0.941 | 0.84 |
| RS3181052\|RS1794066\|RS419598\| | TCAGTAATGCG | 33 | | 0.346 | 1.96 | 0.00944 |
| RS380092\|RS315952\|RS9005\| | TTAGTAATGCG | 34 | | 0.0271 | 1.15 | 0.88 |
| RS315943\|RS137428 | TTAGTATCGTC | 35 | | 0.146 | 0.414 | 0.0307 |
| | TTGACATCGTC | 36 | | 0.0904 | 0.607 | 0.166 |
| | GTGACATCGTC | 37 | | 0.0259 | 1.29 | 0.669 |
| | TCAGTAATATC | 38 | | 0.0124 | 5.42E−10 | 0.999 |
| | GTGGCGATATG | 39 | | 0.014 | 3.04E+09 | 0.999 |
| | TTAGTATTATC | 40 | | 0.0107 | 2.25E−19 | 0.998 |
| | TTAGTATTGCG | 41 | | 0.0188 | 1.10E+262 | 0.994 |
| | TCAGTAATGCC | 42 | | 0.0172 | 0.661 | 0.689 |
| | TTAGCGATATC | 43 | | 0.011 | 4.09 | 0.196 |
| Omnibus model p-value | 0.02243 | | | | | |
| RS315931\|RS4251961\|RS2637988\| | GTGGCGTATC | 44 | 10 | 0.233 | 0.942 | 0.844 |
| RS3181052\|RS1794066\|RS419598\| | TCAGTATGCG | 45 | | 0.345 | 1.96 | 0.00944 |
| RS315952\|RS9005\|RS315943\| | TTAGTATGCG | 46 | | 0.0461 | 1.55 | 0.615 |
| RS137428 | TTAGTACGTC | 47 | | 0.145 | 0.416 | 0.0316 |
| | TTGACACGTC | 48 | | 0.0899 | 0.607 | 0.166 |
| | GTGACACGTC | 49 | | 0.0259 | 1.27 | 0.685 |
| | TCAGTATATC | 50 | | 0.0112 | 5.42E−10 | 0.999 |
| | GTGGCGTATG | 51 | | 0.014 | 3.42E+09 | 0.999 |
| | TTAGTATATC | 52 | | 0.0126 | 2.33E−17 | 0.998 |
| | TCAGTATGCC | 53 | | 0.0172 | 0.661 | 0.689 |
| | TTAGCGTATC | 54 | | 0.011 | 4.06 | 0.198 |
| Omnibus model p-value | 0.01284 | | | | | |
| RS315931\|RS2637988\|RS3181052\| | GGGCGTATC | 55 | 9 | 0.231 | 0.986 | 0.964 |
| RS1794066RS419598\|RS315952\| | TAGTATGCG | 56 | | 0.392 | 1.98 | 0.00714 |
| RS9005\|RS315943\|RS137428 | TAGTACGTC | 57 | | 0.148 | 0.472 | 0.0598 |
| | GGACACGTC | 58 | | 0.0266 | 1.11 | 0.853 |
| | TGACACGTC | 59 | | 0.0925 | 0.579 | 0.132 |
| | GAGTATATC | 60 | | 0.0102 | 0.231 | 0.245 |
| | TAGTATATC | 61 | | 0.0246 | 3.36E−23 | 0.997 |
| | GGGCGTATG | 62 | | 0.0141 | 3.83E+09 | 0.999 |
| | TAGTATGCC | 63 | | 0.0172 | 0.661 | 0.689 |
| | TAGCGTATC | 64 | | 0.0121 | 2.2 | 0.375 |
| Omnibus model p-value | 0.006575 | | | | | |
| RS315931\|RS2637988\|RS3181052\| | GGGCGATC | 65 | 8 | 0.232 | 0.987 | 0.966 |
| RS1794066\|RS419598\|RS9005\| | TAGTAGCG | 66 | | 0.382 | 2.24 | 0.0018 |
| RS315943\|RS137428 | TAGTAGTC | 67 | | 0.16 | 0.385 | 0.0134 |
| | GGACAGTC | 68 | | 0.0278 | 1.13 | 0.837 |
| | TGACAGTC | 69 | | 0.0937 | 0.57 | 0.12 |
| | GAGTAATC | 70 | | 0.0105 | 0.213 | 0.208 |
| | TAGTAACG | 71 | | 0.0119 | 4.94E−10 | 0.999 |
| | TAGTAATC | 72 | | 0.0132 | 1.07E−151 | 0.992 |
| | GGGCGATG | 73 | | 0.0141 | 2.70E+09 | 0.999 |
| | TAGTAGCC | 74 | | 0.0173 | 0.661 | 0.689 |
| | TAGCGATC | 75 | | 0.0123 | 2.18 | 0.38 |
| Omnibus model p-value | 0.003233 | | | | | |
| RS315931\|RS2637988\|RS3181052\| | GGGCGAT | 76 | 7 | 0.246 | 1.05 | 0.864 |
| RS1794066\|RS419598\|RS9005\| | TAGTAGC | 77 | | 0.399 | 2.24 | 0.00205 |
| RS315943 | TAGTAGT | 78 | | 0.161 | 0.387 | 0.0134 |
| | TGACAGT | 79 | | 0.0937 | 0.57 | 0.12 |
| | GGACAGT | 80 | | 0.0277 | 1.13 | 0.831 |
| | GAGTAAT | 81 | | 0.0105 | 0.213 | 0.208 |
| | TAGTAAC | 82 | | 0.0124 | 4.94E−10 | 0.999 |
| | TAGTAAT | 83 | | 0.0133 | 1.82E−152 | 0.992 |
| | TAGCGAT | 84 | | 0.0127 | 1.52 | 0.594 |
| Omnibus model p-value | 0.00227 | | | | | |
| RS315931\|RS3181052\|RS1794066\| | TGTAGC | 85 | 6 | 0.411 | 2.05 | 0.00569 |
| RS419598\|RS9005\|RS315943 | GGCGAT | 86 | | 0.247 | 0.998 | 0.996 |
| | TGTAGT | 87 | | 0.15 | 0.469 | 0.0559 |
| | TACAGT | 88 | | 0.0952 | 0.521 | 0.0741 |
| | TGCGAT | 89 | | 0.0167 | 2.7 | 0.239 |
| | GGTAAT | 90 | | 0.011 | 0.22 | 0.212 |
| | TGTAAT | 91 | | 0.0248 | 0 | 0.978 |
| | GACAGT | 92 | | 0.0264 | 1.25 | 0.709 |
| Omnibus model p-value | 0.002763 | | | | | |
| RS3181052\|RS1794066\|RS419598\| | GTAGC | 93 | 5 | 0.413 | 2.05 | 0.00571 |
| RS9005\|RS315943 | GCGAT | 94 | | 0.263 | 1.17 | 0.603 |
| | GTAGT | 95 | | 0.152 | 0.568 | 0.137 |
| | ACAGT | 96 | | 0.122 | 0.648 | 0.162 |
| | GTAAT | 97 | | 0.0357 | 0.0858 | 0.0238 |
| Omnibus model p-value | 0.0007773 | | | | | |
| RS3181052\|RS419598\|RS9005\| | GAGC | 98 | 4 | 0.413 | 2.05 | 0.00558 |
| RS315943 | GGAT | 99 | | 0.264 | 1.17 | 0.603 |
| | AAGT | 100 | | 0.124 | 0.664 | 0.182 |
| | GAGT | 101 | | 0.152 | 0.566 | 0.135 |
| | GAAT | 102 | | 0.0362 | 0.0853 | 0.0239 |
| Omnibus model p-value | 0.0008699 | | | | | |
| RS419598\|RS9005RS315943 | AGC | | 3 | 0.414 | 2.06 | 0.00553 |
| | AGT | | | 0.276 | 0.568 | 0.033 |
| | GAT | | | 0.264 | 1.17 | 0.603 |
| | AAT | | | 0.0375 | 0.0859 | 0.0238 |
| Omnibus model p-value | 0.0003149 | | | | | |
| RS419598\|RS315943 | AC | | 2 | 0.416 | 2.05 | 0.00571 |
| | AT | | | 0.313 | 0.435 | 0.0018 |
| | GT | | | 0.271 | 1.11 | 0.723 |
| Omnibus model p-value | 0.002012 | | | | | |

TABLE 15b

| | | SEQ | | | |
|---|---|---|---|---|---|
| | | ID | Freq | Freq NON- | |
| SNPs | Haplotype | NO | PROGRESS | PROGRESSORS | P-value |
| RS3181052\|RS1794066\| | GTAGC | 103 | 0.4828 | 0.3205 | 0.004684 |
| RS419598\|RS9005\|RS315943 | GCGAT | 104 | 0.2701 | 0.2423 | 0.586 |
| | GTAGT | 105 | 0.1034 | 0.1648 | 0.1164 |
| | ACAGT | 106 | 0.1379 | 0.1948 | 0.1853 |
| | GTAAT | 107 | 0.005747 | 0.07751 | 0.000964 |
| RS3181052\|RS419598\| | GAGC | 108 | 0.4773 | 0.3177 | 0.005127 |
| RS9005\|RS315943 | GGAT | 109 | 0.267 | 0.2405 | 0.6004 |
| | AAGT | 110 | 0.1477 | 0.2012 | 0.2197 |
| | GAGT | 111 | 0.1023 | 0.1639 | 0.1117 |
| | GAAT | 112 | 0.005682 | 0.0766 | 0.000971 |
| RS419598\|RS9005\|RS315943 | AGC | | 0.4773 | 0.3174 | 0.005035 |
| | AGT | | 0.25 | 0.3653 | 0.02984 |
| | GAT | | 0.267 | 0.2405 | 0.5993 |
| | AAT | | 0.005682 | 0.0769 | 0.000943 |

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive. Variations and changes may be made by those skilled in the art, without departing from the spirit of the invention.

What is claimed is:

1. A method for predicting progression of osteoarthritis in a patient, comprising the steps of:
a. taking a biological sample from said patient;
b. genotyping said biological sample for (i) at least one of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961) and (ii) optionally one or more genetic markers selected from the group consisting of IL1RN (RS3181052), IL1RN (RS1794066), IL1RN (RS419598), IL1RN (RS9005), and IL1RN (RS315943);
c. comparing the genotyping results of step b with a reference;
d. predicting progress of osteoarthritis of said patient based on the patient's genotype.

2. The method of claim 1, wherein the biological sample is genotyped for (i) at least one of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961) and (ii) IL1RN (rs419598) and IL1RN (rs9005).

3. The method of claim 2, wherein the biological sample is genotyped for IL1RN (rs315952), IL1RN (rs419598), and IL1RN (rs9005); wherein a haplotype of rs419598/rs315952/rs9005 (TTA or TCG) indicates that said patient has low risk of osteoarthritis progression; wherein a haplotype of rs419598/rs315952/rs9005 (TTG) indicates that said patient has high risk of osteoarthritis progression.

4. The method of claim 2, wherein the biological sample is genotyped for IL1RN (rs419598), IL1RN (rs9005), and IL1RN (rs315943); wherein a haplotype of rs419598/rs9005/rs315943 (AGT or AAT) indicates that said patient has low risk of osteoarthritis progression; wherein a haplotype of rs419598/rs315952/rs9005 (AGC) indicates that said patient has high risk of osteoarthritis progression.

5. The method of claim 1 wherein said biological sample is genotyped for at least two of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961).

6. The method of claim 4, wherein said biological sample is genotyped for IL1RN (rs3181052), IL1RN (rs 419598), IL1RN (rs9005), and IL1RN (rs315943) wherein a haplotype of RS3181052|RS419598|RS90051RS315943 (GAAT or AAGT) indicates that said patient has low risk of osteoarthritis progression; wherein a haplotype RS3181052|RS419598|RS90051RS315943 (GAGC) indicates that said patient has high risk of osteoarthritis progression.

7. The method of claim 1 wherein said biological sample is genotyped for at least three of the genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL (rs4251961).

8. The method of claim 6, wherein said biological sample is genotyped for IL1RN (rs4251961), IL1RN (rs419598), IL1RN (rs315952), and IL1RN (rs9005); wherein a haplotype of rs4251961/rs419598/rs315952/rs9005 (TTCG) indicates that said patient has low risk of osteoarthritis progression; wherein a haplotype of rs4251961/rs419598/rs315952/rs9005 (CTTG) indicates that said patient has high risk of osteoarthritis progression.

9. The method of claim 6, wherein said biological sample is genotyped for IL1RN RS3181052|RS1794066|RS4195981RS90051 RS315943 wherein a haplotype GTAGT or GTAAT (RS3181052|RS1794066|RS419598|RS90051 RS315943) indicates that said patient has low risk of osteoarthritis progression; wherein a haplotype GTAGC (RS3181052|RS1794066|RS419598|RS90051RS315943) indicates that said patient has high risk of osteoarthritis progression.

10. The method of claim 1 wherein said biological sample is genotyped for (i) at least six genetic markers selected from the group consisting of BMP2 (rs1049007), CLEC3B (rs13963), IL1RN (rs1374281), IL1RN (rs1794066), IL1RN (rs2637988), IL1RN (rs315943), IL1RN (rs315952), IL1RN (rs380092), IL1RN (rs4251961), IL1R1 (rs2287047), IL1R1 (rs315949), VDR (rs10735810), SLC17A3 (rs1165205), OPG (rs2073618), Cilp (rs2073711) and IL1RN (rs4251961); and (ii) at least four of the genetic markers selected from the group consisting of IL1RN (rs419598), IL1RN (rs315931), IL1RN (rs3181052), IL1RN (rs579543) and IL1RN (rs9005).

11. The method of claim 1, further comprising the identification of an IL1RN haplotype which comprises at least seven markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943) and IL1RN(rs1374281); wherein said IL1RN haplotype with at least seven markers can be used to predict whether said patient is at high risk, neutral or low risk from OA progression.

12. The method of claim 10, wherein said IL1RN haplotype comprises at least seven markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), and IL1RN(rs1374281).

13. The method of claim 10, wherein said IL1RN haplotype comprises at least ten markers selected from the group consisting of IL1RN(rs315931), IL1RN(rs4251961), IL1RN(rs2637988), IL1RN(rs3181052), IL1RN(rs1794066), IL1RN(rs419598), ILIRN(rs380092), IL1RN(rs579543), IL1RN(rs315952), IL1RN(rs9005), IL1RN(rs315949), IL1RN(rs315943), and IL1RN(rs1374281).

14. The method of claim 10, wherein said IL1RN haplotype comprises at least ten markers selected from the group consisting of IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), and IL1RN(rs1374281).

15. The method of claim 13, wherein a haplotype of TCAGTAACTGCG (rs315931/rs4251961/rs2637988/rs3181052/rs1794066/rs419598/rs380092/rs579543/rs3159 52/rs9005/rs315943/rs1374281) indicate that said human subject is at risk of osteoarthritis progression; a haplotype of GTGGCGATTATC (rs315931/rs4251961/rs2637988/rs3181052/rs1794066/rs419598/rs380092/rs579543/rs3159 52/rs9005/rs315943/rs1374281) indicates that said human subject is neutral to osteoarthritis progression; and a haplotype of TTAGTATCCGTC (rs315931/rs4251961/rs2637988/rs3181052/rs1794066/rs419598/rs380092/rs579543/rs3159 52/rs9005/rs315943/rs1374281) indicates that said human subject is at low risk of osteoarthritis progression.

16. The method of claim 10, wherein said IL1RN haplotype comprises IL1RN(rs315931), IL1RN(rs4251961), IL1RN(rs2637988), IL1RN(rs3181052), IL1RN(rs1794066), IL1RN(rs419598), IL1RN(rs380092), IL1RN(rs579543), IL1RN(rs315952), IL1RN(rs9005), IL1RN(rs315949), IL1RN(rs315943), IL1RN(rs1374281).

17. A method of predicting progression of osteoarthritis comprising the steps of claim 1 and further comprising the step of calculating a gene score of said patient to predict severity of osteoarthritis progression.

18. The method of claim 17, wherein a gene score of 2 or less indicates that such patient is at very low risk; wherein a gene score of 3-4 indicates that the patient is at low risk of osteoarthristis progression; wherein a gene score of 5-6 indicates that the patient is at risk of osteoarthristis progression; wherein a gene score of 7 or above indicates that the patient is at high risk of osteoarthristis progression.

19. The method of claim 1, wherein said biological sample is saliva, buccal cells, blood, tissue samples or urine.

20. A method for predicting initiation of osteoarthritis in a patient, comprising the steps of:
a. taking a biological sample from said patient;
b. genotyping said biological sample for at least one of the genetic markers selected from the group consisting of ADAM12 (rs3740199), BMP2 (rs1049007), CLEC3B (rs13963), HFE (rs1799945), IL1RN (rs315931), IL1RN (rs419598), IL1RN (rs579543), IL1RN (rs9005), IL1B(rs1143623), ADAM12(rs1871054), OPG(rs2073618), IL1RN(rs315943), IL1RN(rs315949), IL1RN(rs4251961), CDC42BPB(rs751837) and IL1RN(rs315952).
c. comparing the genotyping results of step b with a reference;
d. predicting said patient's risk of osteoarthritis initiation based on said patient's genotype.

21. The method of claim 20, wherein said biological sample is genotyped for at least two of the genetic markers selected from the group consisting of ADAM12 (rs3740199), BMP2 (rs1049007), CLEC3B (rs13963), HFE (rs1799945), IL1RN (rs315931), IL1RN (rs419598), IL1RN (rs579543), IL1RN (rs9005), IL1B(rs1143623), ADAM12(rs1871054), OPG(rs2073618), IL1RN(rs315943), IL1RN(rs315949), IL1RN(rs4251961), CDC42BPB(rs751837) and IL1RN(rs315952).
